(19) United States
(12) Patent Application Publication
(10) Pub. No.: US 2023/0346284 A1
(43) Pub. Date: Nov. 2, 2023

(54) Dry Electrode with Sliding Electroconductive Protrusions for Use on a Hair-Covered Area of a Person's Head

(71) Applicant: Robert A. Connor, St. Paul, MN (US)

(72) Inventor: Robert A. Connor, St. Paul, MN (US)

(73) Assignee: Medibotics LLC, St. Paul, MN (US)

(21) Appl. No.: 18/219,684

(22) Filed: Jul. 9, 2023

Related U.S. Application Data
(63) Continuation-in-part of application No. 17/714,988, filed on Apr. 6, 2022.
Application No. 17/714,988 is a continuation-in-part of application No. 16/838,541, filed on Apr. 2, 2020.
Application No. 17/714,988 is a continuation-in-part of application No. 17/665,086, filed on Feb. 4, 2022, now Pat. No. 11,662,819.
Application No. 17/665,086 is a continuation-in-part of application No. 17/136,117, filed on Dec. 29, 2020, now abandoned.
Application No. 17/136,117 is a continuation-in-part of application No. 16/554,029, filed on Aug. 28, 2019, now abandoned.
Application No. 17/665,086 is a continuation-in-part of application No. 17/136,117, filed on Dec. 29, 2020, now abandoned.
Application No. 17/136,117 is a continuation-in-part of application No. 16/554,029, filed on Aug. 28, 2019, now abandoned.
Application No. 17/136,117 is a continuation-in-part of application No. 16/838,541, filed on Apr. 2, 2020.
Application No. 16/838,541 is a continuation-in-part of application No. 16/737,052, filed on Jan. 8, 2020, now Pat. No. 11,754,542.
Application No. 16/737,052 is a continuation-in-part of application No. 16/568,580, filed on Sep. 12, 2019, now Pat. No. 11,478,158.
Application No. 16/568,580 is a continuation-in-part of application No. 16/554,029, filed on Aug. 28, 2019, now abandoned.
Application No. 16/554,029 is a continuation-in-part of application No. 16/554,029, filed on Aug. 28, 2019, now abandoned.
Application No. 16/554,029 is a continuation-in-part of application No. 15/236,401, filed on Aug. 13, 2016, now abandoned.
Application No. 16/737,052 is a continuation-in-part of application No. 16/568,580, filed on Sep. 12, 2019, now Pat. No. 11,478,158.
Application No. 16/568,580 is a continuation-in-part of application No. 15/963,061, filed on Apr. 25, 2018, now Pat. No. 10,772,559.
Application No. 16/568,580 is a continuation-in-part of application No. 15/963,061, filed on Apr. 25, 2018, now Pat. No. 10,772,559.
Application No. 15/963,061 is a continuation-in-part of application No. 16/022,987, filed on Jun. 29, 2018, now Pat. No. 11,172,859.
Application No. 16/022,987 is a continuation-in-part of application No. 15/136,948, filed on Apr. 24, 2016, now Pat. No. 10,234,942.
Application No. 15/963,061 is a continuation-in-part of application No. 15/464,349, filed on Mar. 21, 2017, now Pat. No. 9,968,297.
Application No. 15/464,349 is a continuation-in-part of application No. 15/136,948, filed on Apr. 24, 2016, now Pat. No. 10,234,942.
Application No. 15/136,948 is a continuation-in-part of application No. 14/562,719, filed on Dec. 7, 2014, now Pat. No. 10,130,277.
Application No. 14/562,719 is a continuation-in-part of application No. 14/330,649, filed on Jul. 14, 2014, now abandoned.
Application No. 15/236,401 is a continuation-in-part of application No. 15/136,948, filed on Apr. 24, 2016, now Pat. No. 10,234,942.
Application No. 15/136,948 is a continuation-in-part of application No. 14/599,522, filed on Jan. 18, 2015, now Pat. No. 9,814,426.
Application No. 14/599,522 is a continuation-in-part of application No. 14/599,522, filed on Jan. 18, 2015, now Pat. No. 9,814,426.
Application No. 14/599,522 is a continuation-in-part of application No. 14/562,719, filed on Dec. 7, 2014, now Pat. No. 10,130,277.
Application No. 14/330,649 is a continuation-in-part of application No. 13/797,955, filed on Mar. 12, 2013, now Pat. No. 9,456,916.
Application No. 13/797,955 is a continuation-in-part of application No. 13/523,739, filed on Jun. 14, 2012, now Pat. No. 9,042,596.
(60) Provisional application No. 62/972,692, filed on Feb. 11, 2020.
(60) Provisional application No. 62/972,692, filed on Feb. 11, 2020.
(60) Provisional application No. 62/851,917, filed on May 23, 2019.
(60) Provisional application No. 62/837,712, filed on Apr. 23, 2019.
(60) Provisional application No. 62/851,904, filed on May 23, 2019.
(60) Provisional application No. 62/796,901, filed on Jan. 25, 2019.
(60) Provisional application No. 62/791,838, filed on Jan. 13, 2019.
(60) Provisional application No. 62/430,667, filed on Dec. 6, 2016.
(60) Provisional application No. 62/322,594, filed on Apr. 14, 2016.
(60) Provisional application No. 62/303,126, filed on Mar. 3, 2016.
(60) Provisional application No. 62/169,661, filed on Jun. 2, 2015.
(60) Provisional application No. 62/160,172, filed on May 12, 2015.
(60) Provisional application No. 62/089,696, filed on Dec. 9, 2014.
(60) Provisional application No. 62/017,615, filed on Jun. 26, 2014.
(60) Provisional application No. 61/939,244, filed on Feb. 12, 2014.
(60) Provisional application No. 61/932,517, filed on Jan. 28, 2014.
(60) Provisional application No. 61/932,517, filed on Jan. 28, 2014.
(60) Provisional application No. 61/729,494, filed on Nov. 23, 2012.

(51) Int. Cl. A61B 5/291; A61B 5/00

(52) U.S. Cl. CPC A61B 5/291; A61B 5/6814

(57) ABSTRACT

This invention is a dry electrode for use on a hair-covered area of a person's head to record EEG signals or deliver neurostimulation. This dry electrode has a plurality of electroconductive protrusions which extend out from a base toward the surface of a person's head. These protrusions slide through hair without being so narrow that they do not get good electrical contact and without being so stiff that they are uncomfortable for extended wear.

### CROSS-REFERENCE TO RELATED APPLICATIONS

This patent application is a continuation-in-part of U.S. patent application Ser. No. 17/714,988 filed on 2022 Apr. 6. This patent application is a continuation-in-part of U.S. patent application Ser. No. 16/838,541 filed on 2020 Apr. 2.

U.S. patent application Ser. No. 17/714,988 was a continuation-in-part of U.S. patent application Ser. No. 17/665,086 filed on 2022 Feb. 4. U.S. patent application Ser. No. 17/714,988 was a continuation-in-part of U.S. patent application Ser. No. 17/136,117 filed on 2020 Dec. 29. U.S. patent application Ser. No. 17/714,988 was a continuation-in-part of U.S. patent application Ser. No. 16/554,029 filed on 2019 Aug. 28. U.S. patent application Ser. No. 17/665,086 was a continuation-in-part of U.S. patent application Ser. No. 17/136,117 filed on 2020 Dec. 29. U.S. patent application Ser. No. 17/665,086 was a continuation-in-part of U.S. patent application Ser. No. 16/554,029 filed on 2019 Aug. 28.

U.S. patent application Ser. No. 17/136,117 was a continuation-in-part of U.S. patent application Ser. No. 16/838,541 filed on 2020 Apr. 2. U.S. patent application Ser. No. 17/136,117 claimed the priority benefit of U.S. provisional patent application 62/972,692 filed on 2020 Feb. 11. U.S. patent application Ser. No. 17/136,117 was a continuation-in-part of U.S. patent application Ser. No. 16/737,052 filed on 2020 Jan. 8. U.S. patent application Ser. No. 17/136,117 was a continuation-in-part of U.S. patent application Ser. No. 16/568,580 filed on 2019 Sep. 12. U.S. patent application Ser. No. 17/136,117 was a continuation-in-part of U.S. patent application Ser. No. 16/554,029 filed on 2019 Aug. 28.

U.S. patent application Ser. No. 16/838,541 claimed the priority benefit of U.S. provisional patent application 62/972,692 filed on 2020 Feb. 11. U.S. patent application Ser. No. 16/838,541 was a continuation-in-part of U.S. patent application Ser. No. 16/554,029 filed on 2019 Aug. 28. U.S. patent application Ser. No. 16/838,541 claimed the priority benefit of U.S. provisional patent application 62/851,917 filed on 2019 May 23. U.S. patent application Ser. No. 16/838,541 claimed the priority benefit of U.S. provisional patent application 62/837,712 filed on 2019 Apr. 23. U.S. patent application Ser. No. 16/838,541 was a continuation-in-part of U.S. patent application Ser. No. 15/236,401 filed on 2016 Aug. 13. U.S. patent application Ser. No. 16/737,052 was a continuation-in-part of U.S. patent application Ser. No. 16/568,580 filed on 2019 Sep. 12. U.S. patent application Ser. No. 16/737,052 was a continuation-in-part of U.S. patent application Ser. No. 15/963,061 filed on 2018 Apr. 25. U.S. patent application Ser. No. 16/568,580 was a continuation-in-part of U.S. patent application Ser. No. 15/963,061 filed on 2018 Apr. 25.

U.S. patent application Ser. No. 16/554,029 claimed the priority benefit of U.S. provisional patent application 62/851,904 filed on 2019 May 23. U.S. patent application Ser. No. 16/554,029 claimed the priority benefit of U.S. provisional patent application 62/796,901 filed on 2019 Jan. 25. U.S. patent application Ser. No. 16/554,029 claimed the priority benefit of U.S. provisional patent application 62/791,838 filed on 2019 Jan. 13. U.S. patent application Ser. No. 16/554,029 was a continuation-in-part of U.S. patent application Ser. No. 16/022,987 filed on 2018 Jun. 29. U.S. patent application Ser. No. 16/022,987 was a continuation-in-part of U.S. patent application Ser. No. 15/136,948 filed on 2016 Apr. 24. U.S. patent application Ser. No. 15/963,061 was a continuation-in-part of U.S. patent application Ser. No. 15/464,349 filed on 2017 Mar. 21.

U.S. patent application Ser. No. 15/464,349 claimed the priority benefit of U.S. provisional patent application 62/430,667 filed on 2016 Dec. 6. U.S. patent application Ser. No. 15/464,349 was a continuation-in-part of U.S. patent application Ser. No. 15/136,948 filed on 2016 Apr. 24. U.S. patent application Ser. No. 15/464,349 was a continuation-in-part of U.S. patent application Ser. No. 14/562,719 filed on 2014 Dec. 7. U.S. patent application Ser. No. 15/464,349 was a continuation-in-part of U.S. patent application Ser. No. 14/330,649 filed on 2014 Jul. 14. U.S. patent application Ser. No. 15/236,401 was a continuation-in-part of U.S. patent application Ser. No. 15/136,948 filed on 2016 Apr. 24. U.S. patent application Ser. No. 15/236,401 was a continuation-in-part of U.S. patent application Ser. No. 14/599,522 filed on 2015 Jan. 18.

U.S. patent application Ser. No. 15/136,948 claimed the priority benefit of U.S. provisional patent application 62/322,594 filed on 2016 Apr. 14. U.S. patent application Ser. No. 15/136,948 claimed the priority benefit of U.S. provisional patent application 62/303,126 filed on 2016 Mar. 3. U.S. patent application Ser. No. 15/136,948 claimed the priority benefit of U.S. provisional patent application 62/169,661 filed on 2015 Jun. 2. U.S. patent application Ser. No. 15/136,948 claimed the priority benefit of U.S. provisional patent application 62/160,172 filed on 2015 May 12. U.S. patent application Ser. No. 15/136,948 was a continuation-in-part of U.S. patent application Ser. No. 14/599,522 filed on 2015 Jan. 18.

U.S. patent application Ser. No. 14/599,522 claimed the priority benefit of U.S. provisional patent application 62/089,696 filed on 2014 Dec. 9. U.S. patent application Ser. No. 14/599,522 was a continuation-in-part of U.S. patent application Ser. No. 14/562,719 filed on 2014 Dec. 7. U.S. patent application Ser. No. 14/599,522 claimed the priority benefit of U.S. provisional patent application 62/017,615 filed on 2014 Jun. 26. U.S. patent application Ser. No. 14/599,522 claimed the priority benefit of U.S. provisional patent application 61/939,244 filed on 2014 Feb. 12. U.S. patent application Ser. No. 14/599,522 claimed the priority benefit of U.S. provisional patent application 61/932,517 filed on 2014 Jan. 28.

U.S. patent application Ser. No. 14/562,719 claimed the priority benefit of U.S. provisional patent application 61/932,517 filed on 2014 Jan. 28. U.S. patent application Ser. No. 14/330,649 was a continuation-in-part of U.S. patent application Ser. No. 13/797,955 filed on 2013 Mar. 12. U.S. patent application Ser. No. 14/330,649 was a continuation-in-part of U.S. patent application Ser. No. 13/523,739 filed on 2012 Jun. 14. U.S. patent application Ser. No. 13/797,955 claimed the priority benefit of U.S. provisional patent application 61/729,494 filed on 2012 Nov. 23.

The entire contents of these applications are incorporated herein by reference.

### FEDERALLY SPONSORED RESEARCH

Not Applicable

### SEQUENCE LISTING OR PROGRAM

Not Applicable

### BACKGROUND—FIELD OF INVENTION

### Introduction

There are numerous potential applications for incorporating EEG electrodes into wearable technology such as smart eyewear, smart earwear, smart headbands, and smart adhesive patches. However, wet EEG electrodes are not well-suited for use with such wearable technology and flat dry EEG electrodes do not work well on areas of a person's head which are covered by hair. There has been some creative innovation in the prior art in the development of dry electrodes with protrusions which penetrate between strands of hair. However, protrusions in the prior art which are sufficiently resilient and narrow to penetrate hair can be uncomfortable, cause skin irritation, and lack sufficient contact area to enable good electromagnetic communication with a person's head. There remains an unmet need for dry EEG electrodes which can be incorporated into wearable technology and work well on areas of a person's head which are covered by hair.

### Review of the Relevant Art

There is creative innovation underway with respect to dry EEG electrodes. In the patent literature, U.S. patent application 20090134887 (Hu et al., May 28, 2009, “Contact Sensor”) discloses a sensor with a main body and arc-shaped conductors. U.S. patent application 20130274583 (Heck, Oct. 17, 2013, “Electrodes Adapted for Transmitting or Measuring Voltages Through Hair”) discloses a composite metal electrode with raised points which pass through hair to contact skin. U.S. patent applications 20140024912 (Dalke, Jan. 23, 2014, “Neurophysiological Dry Sensor”) and 20150265176 (Dalke, Sep. 24, 2015, “Neurophysiological Dry Sensor”) disclose a sensor with conductive spires. U.S. patent application 20140107458 (Op De Beeck et al., Apr. 17, 2014, “Resilient Sensor for Biopotential Measurements”) discloses a sensor with a cavity wherein an electrical contacting unit is partially secured.

U.S. Pat. No. 8,798,710 (Chi, Aug. 5, 2014, “Apparatuses, Systems and Methods for Biopotential Sensing with Dry Electrodes”) discloses an electrode with an electrical conductor, a membrane permeable to ionic conduction, and a conductive medium in communication with a portion of the electrical conductor and a portion of the membrane. U.S. patent application 20140288406 (Chai, Sep. 25, 2014, “Line-Contact Dry Electrode”) discloses a line-contact dry electrode with elastic conductive branches forming a comb-like electrode. U.S. patent application 20150088224 (Goldwasser et al., Mar. 26, 2015, “Wearable Transdermal Electrical Stimulation Devices and Methods of Using Them”) discloses an electrode with first and second transdermal electrodes. U.S. patent application 20150141788 (Chi et al., May 21, 2015, “Transducer Assemblies for Dry Applications of Transducers”) and U.S. Pat. No. 9,314,183 (Chi et al., Apr. 19, 2016, “Transducer Assemblies for Dry Applications of Transducers”) disclose at least one probe extending from a terminal to penetrate through patches of hair.

U.S. patent application 20150367122 (Morshed et al., Dec. 24, 2015, “Patterned Carbon Nanotube Electrode”) discloses an electrode with carbon nanotube pillars. U.S. patent application 20160089045 (Sadeghian-Motahar et al., Mar. 31, 2016, “Bio-Potential Sensing Materials as Dry Electrodes and Devices”) discloses a dry electrode in contact with skin to receive bio-potential signals. U.S. patent application 20160143554 (Lim et al., May 26, 2016, “Apparatus for Measuring Bioelectrical Signals”) discloses a sensor electrode with a tapering portion and a protruding portion that extends from the tapering portion. U.S. patent application 20160174859 (Oudenhoven et al., Jun. 23, 2016, “Electrode for Biopotential Sensing”) discloses an electrode base and a plurality of mesh pins protruding from the electrode base.

U.S. patent application 20170112444 (Lin et al., Apr. 27, 2017, “Bio-Signal Sensor”) discloses a dry electrode with probes and contacts correspondingly and electrically connected to the probes, wherein each of the probes senses and transmits an electrical signal to the corresponding contact. U.S. patent application 20170150925 (Jung, Jun. 1, 2017, “EEG Hair Band”) discloses EEG sensors embodied in a hair band. U.S. patent application 20170172447 (Mitra et al., Jun. 22, 2017, “Sensor, System, and Holder Arrangement for Biosignal Activity Measurement”) discloses a sensor with a pins protruding from an electrode base. U.S. patent application 20170224990 (Goldwasser et al., Aug. 10, 2017, “Apparatuses and Methods for Neuromodulation”) discloses apparatuses and methods to apply an ensemble current waveform between two or more electrodes.

U.S. patent application 20170258353 (Jovanovic et al., Sep. 14, 2017, “Headsets and Electrodes for Gathering Electroencephalographic Data”) discloses electrodes with a housing, a spring, and a pin. U.S. patent application 20170258400 (Jovanovic et al., Sep. 14, 2017, “Headsets and Electrodes for Gathering Electroencephalographic Data”) discloses an electrode with a ring disposed in an opening and an arm, where the arm has a first portion extending outward from the opening away from the housing and a second portion extending from an end of the first portion toward the housing and into the cavity, and the first and second portions connect at a bend.

U.S. Pat. No. 9,820,670 (Parvizi et al., Nov. 21, 2017, “Methods and Apparatus for Electrode Placement and Tracking”) and 10888240 (Parvizi et al., Jan. 12, 2021, “Methods and Apparatus for Electrode Placement and Tracking”) as well as U.S. patent applications 20220117536 (Parvizi et al., Apr. 21, 2022, “Methods and Apparatus for Electrode Placement and Tracking”) and 20220117535 (Parvizi et al., Apr. 21, 2022, “Methods and Apparatus for Electrode Placement and Tracking”) disclose tubular members extending from an electrode body. U.S. patent application 20180049639 (Tian, Feb. 22, 2018, “Dry Electrode, Its Manufacturing Method and Bio-Electromagnetic Wave Detecting Device and Sensor Element Comprising the Dry Electrode”) discloses a dry electrode with a protruding structure comprising an inner core made of a flexible insulating material and a conductive thin film coated on an outer side of the inner core.

U.S. patent applications 20180153470 (Gunasekar et al., Jun. 7, 2018, “Electroencephalography Headset and System for Collecting Biosignal Data”), 20200281527 (Gunasekar et al., Sep. 10, 2020, “Electroencephalography Headset and System for Collecting Biosignal Data”), 20220015701 (Gunasekar et al., Jan. 20, 2022, “Electroencephalography Headset and System for Collecting Biosignal Data”), and 20220022813 (Gunasekar et al., Jan. 27, 2022, “Electroencephalography Headset and System for Collecting Biosignal Data”) disclose a system for collecting biosignal data including: a first electrode fixedly mounted to a first band and centered between left and right junctions; a second electrode mounted to the first band offset from the first electrode and laterally-adjustable along the length of the first band; and a third electrode mounted to a second band and laterally-adjustable along the length of the second band.

U.S. patent application 20180192906 (Soulet De Brugiere et al., Jul. 12, 2018, “Polymer Composition and Electrode for a Device for the Non-Invasive Measurement of Biological Electrical Signals”) discloses a polymer matrix in which are dispersed carbon nanotubes and adsorbent elements selected from activated carbon particles and graphene nanoplatelets. U.S. patent application 20180235499 (Zorman et al., Aug. 23, 2018, “Method for Measuring an Electrophysiological Parameter by Means of a Capacitive Electrode Sensor of Controlled Capacitance”) discloses a sensor with a base and a plurality of protrusions projecting from the base. U.S. patent application 20180235500 (Lee et al., Aug. 23, 2018, “Dry Electrode for Detecting Biosignal and Method for Manufacturing Same”) discloses a dry electrode comprising a protrusion and a coating on an end surface of the protrusion.

U.S. patent applications 20180348863 (Aimone et al., Dec. 6, 2018, “Wearable Computing Device With Electrophysiological Sensors”), 20200019243 (Aimone et al., Jan. 16, 2020, “Wearable Computing Device With Electrophysiological Sensors”), and 20210200313 (Aimone et al., Jul. 1, 2021, “Wearable Computing Device With Electrophysiological Sensors”) disclose a wearable computing device with bio-signal sensors and a feedback module providing an interactive mediated reality (“VR”) environment. U.S. patent application 20180353096 (Mercier et al., Dec. 13, 2018, “Electrode, Wearable Assembly and System”) and U.S. patent Ser. No. 10/842,404 (Mercier et al., Nov. 24, 2020, “Electrode, Wearable Assembly and System”) disclose an electrode with a base and a plurality of legs extending from the base. U.S. patent application 20190000338 (Van Den Ende et al., Jan. 3, 2019, “Method and System for Obtaining Signals from Dry EEG Electrodes”) discloses an actuator coupled to an EEG electrode and configured to move the electrode in at least two dimensions.

U.S. patent Ser. No. 10/285,646 (Grant et al., May 14, 2019, “Connection Quality Assessment for EEG Electrode Arrays”) and 10980480 (Grant et al., Apr. 20, 2021, “Connection Quality Assessment for EEG Electrode Arrays”) disclose systems, devices, and methods to assess connection quality between the electrodes of a bioelectrical signal measurement and/or electrical stimulation device and the tissue, typically skin, of a subject. U.S. patent application 20190200925 (Aimone et al., Jul. 4, 2019, “Wearable Computing Device”) discloses a wearable device including a flexible band having at least a front portion to contact at least part of a frontal region of a user's head, a rear portion to contact at least part of an occipital region of the user's head, and at least one side portion extending between the front portion and the rear portion to contact at least part of an auricular region of the user's head. U.S. patent application 20190239807 (Watson et al., Aug. 8, 2019, “Hair Ratcheting Electroencephalogram Sensors”) discloses a sensor with a locking mechanism which permits one-way axial motion of a thread through a channel from the first side to the second side. U.S. patent application 20190328261 (Shakour et al., Oct. 31, 2019, “Brush Electrode”) discloses a plurality of strand electrodes extending outward from an electrode base.

U.S. patent applications 20190365270 (Bachelder et al., Dec. 5, 2019, “Adjustable Geometry Wearable Electrodes”) and 20230000416 (Bachelder et al., Jan. 5, 2023, “Adjustable Geometry Wearable Electrodes”) as well as U.S. patent Ser. No. 10/433,756 (Bachelder et al., Oct. 8, 2019, “Adjustable Geometry Wearable Electrodes”) and U.S. Pat. No. 11,357,434 (Bachelder et al., Jun. 14, 2022, “Adjustable Geometry Wearable Electrodes”) disclose electrode assemblies which contact a patient's scalp and hair via collapse, compression, or telescoping. U.S. patent application 20200069206 (Zaliasl et al., Mar. 5, 2020, “System and a Method for Acquiring an Electrical Signal and a Wearable Device”) discloses a system for acquiring an electrical signal comprising: a plurality of electrodes, a plurality of signal quality detectors, each detector, being configured to detect a signal from a pair of electrodes and each detector comprising an analog-to-digital converter. U.S. patent applications 20200159324 (Keller et al., May 21, 2020, “Headware for Computer Control”) and 20210109594 (Keller et al., Apr. 15, 2021, “Headware for Computer Control”) disclose headware configured to sit at the top of a head and apply pressure to at least one side of the head.

U.S. patent application 20200237249 (Gunasekar et al., Jul. 30, 2020, “Headset and Electrodes for Sensing Bioelectrical Potential and Methods of Operation Thereof”) discloses an electrode tip body, one or more deflectable electrode legs coupled to the electrode tip body, and a conductive cushioning material coupled to a segment of at least one of one or more electrode legs. U.S. patent Ser. No. 10/743,809 (Kamousi et al., Aug. 18, 2020, “Systems and Methods for Seizure Prediction and Detection”) discloses using a machine learning algorithm to perform a seizure binary classification. U.S. patent applications 20200305786 (Grant et al., Oct. 1, 2020, “Systems and Methods for Processing Sonified Brain Signals”) and 20210267539 (Grant et al., Sep. 2, 2021, “Systems and Methods for Processing Sonified Brain Signals”) as well as U.S. patent Ser. No. 10/849,553 (Grant et al., Dec. 1, 2020, “Systems and Methods for Processing Sonified Brain Signals”) disclose systems and methods for sonifying electrical signals, particularly EEG signals.

U.S. patent application 20210038106 (Ramakrishnan et al., Feb. 11, 2021, “Mobile, Wearable EEG Device With High Quality Sensors”) discloses sensor units with conductive segments in a flexible sensing layer. U.S. patent application 20210282695 (Goldstein et al., Sep. 16, 2021, “Personal Apparatus for Conducting Electroencephalography”) discloses an apparatus for conducting electroencephalography allowing for secure and easy application to a human subject's forehead. U.S. patent application 20210338128 (Le Lous et al., Nov. 4, 2021, “Sensor for Measuring a Biological Potential”) discloses an electrode with a base and at least one leg. U.S. patent application 20210353200 (Xu et al., Nov. 18, 2021, “Electrode for Potential Acquisition of a Surface and Manufacturing Method Thereof”) discloses an electrode with at least two pins.

U.S. patent application 20220000407 (Ludwig et al., Jan. 6, 2022, “Dry Electrodes”) discloses an electrode with a substrate, electrically conductive particles in contact with the substrate, a supporting layer that envelopes the electrically conductive particles with points that protrude from the supporting layer, and an electrical connector. U.S. patent application 20220004257 (Keller et al., Jan. 6, 2022, “Headware for Computer Control”) discloses headware with a first arm pivotally coupled to a body portion and a second arm pivotably coupled to the body portion. U.S. patent application 20220211313 (Lee, Jul. 7, 2022, “Dry Electroencephalographic Electrode”) discloses a dry EEG electrode with a plurality of metal probes. U.S. patent Ser. No. 11/471,088 (Parvizi et al., Oct. 18, 2022, “Handheld or Wearable Device for Recording or Sonifying Brain Signals”) discloses a handheld device for sonifying electrical signals obtained from a subject.

U.S. patent application 20230031613 (Fleury, Feb. 2, 2023, “Wearable Device”) discloses a wearable device with a flexible retention mount to allow rotation of a flexible and extendable body relative to an electronics module and to transfer tension force from the flexible and extendable body to the electronics module. U.S. patent application 20230043938 (Kele, Feb. 9, 2023, “Flexible Electroencephalography Headset”) discloses an electrode tip with a thin conductive probe mounted on an elastic beam to extend from a base of the electrode tip, bypass hair, and electrically couple to the head of the user. U.S. patent application 20230165503 (Coyle, Jun. 1, 2023, “Flexible Electrical Measurement Apparatus”) discloses an electrode with a central portion and a plurality of legs extending radially outwards from the central portion.

There is also relevant art in the non-patent literature. (Acar, 2019), “Wearable and Flexible Textile Electrodes for Biopotential Signal Monitoring: A Review,” Electronics, 2019, 8(5), 479, presents a systematic review of wearable textile electrodes for physiological signal monitoring. (Casson, 2019), “Wearable EEG and Beyond,” Biomedical Engineering Letters, January, 2019, 9(1), 53-71, reviews recent progress on electrodes used to make connections to the head and the physical EEG hardware. (Chen, 2014), “Soft, Comfortable Polymer Dry Electrodes for High Quality ECG and EEG Recording,” Sensors, Dec. 10, 2014, 14(12), 23758-80, discloses dry electrodes fabricated from EPDM rubber containing various additives for optimum conductivity, flexibility and ease of fabrication.

(Chen, 2016), “Polymer-Based Dry Electrodes for Biopotential Measurements,” Thesis, Arenberg Doctoral School, 2016, investigates the mechanical properties of the polymer dry electrodes with compression tests for elastic modulus and compliance characterization. (Chi, 2010), “Dry-Contact and Noncontact Biopotential Electrodes: Methodological Review,” IEEE Reviews in Biomedical Engineering, 2010, 3, 106-119, explores the use of dry/noncontact electrodes for clinical use by explaining the electrical models for dry, insulated and noncontact electrodes and showing performance limits, along with measured data. (Chlaihawi, 2018), “Development of Printed and Flexible Dry ECG Electrodes,” Sensing and Bio-Sensing Research, 2018, 20, 9-15, discloses printed, flexible and wearable dry electrodes for monitoring electrocardiogram (ECG) signals without any skin preparation or wet gel.

(Flumeri, 2019), “The Dry Revolution: Evaluation of Three Different EEG Dry Electrode Types in Terms of Signal Spectral Features, Mental States Classification and Usability,” Sensors, Mar. 19, 2019, 19(6), 1365, compares three different dry electrode types: gold-coated single pin, multiple pins and solid-gel. (Fu, 2020), “Dry Electrodes for Human Bioelectrical Signal Monitoring,” Sensors, 6/29/2020, 20(13), 3651, gives a retrospective overview of the development of dry electrodes used for monitoring bioelectrical signals, including sensing principles, material selection, device preparation, and measurement performance. (Gao, 2018), “Soft Pin-Shaped Dry Electrode with Bristles for EEG Signal Measurements,” Sensors and Actuators, 2018, Vol. 283, 348-361, presents a novel soft pin-shaped dry electrode for electroencephalography recording.

(Hsu, 2014), “Developing Barbed Microtip-Based Electrode Arrays for Biopotential Measurement,” Sensors, 2014, 14(7), 12370-12386, discloses the fabrication of barbed microtip-based electrode arrays via silicon wet etching. (Kocturova, 2019), “Comparison of Dry Electrodes for Mobile EEG System,” 2019, evaluates two types of comb electrodes: one based on a Ag—AgCl alloy and one based on a flexible conductive polymer. (Krachunov, 2016), “3D Printed Dry EEG Electrodes,” Sensors, 2016, 16(10), 1635, presents a novel methodology for the design and manufacture of dry electrodes using low cost desktop 3D printers.

(Lau-Zhu, 2019), “Mobile EEG in Research on Neurodevelopmental Disorders: Opportunities and Challenges,” Developmental Cognitive Neuroscience, 2019, Vol. 36, presents a brief overview of recent developments in mobile EEG technologies. (Lee, 2015), “Reverse-Curve-Arch-Shaped Dry EEG Electrode for Increased Skin-Electrode Contact Area on Hairy Scalps,” Electronics Letters, Oct. 1, 2015, discloses reverse-curve-arch-shaped dry EEG electrodes for use in increasing the skin-electrode contact area on hairy scalps. (Lopez-Gordo, 2014), “Dry EEG Electrodes,” Sensors, Jul. 18, 2014, 14(7), 12847-70, reviews current approaches to developing dry EEG electrodes for clinical and other applications.

(Mota, 2013), “Development of a Quasi-Dry Electrode for EEG Recording,” Sensors and Actuators, 2013, Vol. 199, 310-317, reports on the development of a novel polymer-based electrode prototype for electroencephalography (EEG) between classic “wet” and “dry” electrodes. (Olesen, 2020), “Development and Assessment of Electrodes and Instrumentation for Plantar Skin Impedance Measurements,” Thesis, Master in Electronics, Informatics and Technology, University of Oslo, Autumn, 2020, describes the development and testing of electrodes for plantar bioimpedance measurements. (Ouyang, 2021), “Application of Intrinsically Conducting Polymers in Flexible Electronics,” SmartMat, 8/18/2021, 2, discusses the use of intrinsically conducting polymers (ICPs), such as polyacetylene, polyaniline, polypyrrole, polythiophene, and poly(3,4-ethylenedioxythiophene) (PEDOT) for dry electrodes.

(Ruffini, 2008), “First Human Trials of a Dry Electrophysiology Sensor Using a Carbon Nanotube Array Interface,” Sensors and Actuators, Jun. 15, 2008, 144, reports the results from the first human trials of a new dry electrode sensor for surface biopotential applications, wherein the contact surface of the electrode is covered with carbon nanotubes. (Shad, 2020), “Impedance and Noise of Passive and Active Dry EEG Electrodes: A Review,” IEEE Sensors Journal, 7/27/2020, reviews the impedance and noise of passive and active dry EEG electrodes. (Sunwoo, 2020), “Advances in Soft Bioelectronics for Brain Research and Clinical Neuroengineering,” Matter, 2020, 3(6) 1923-1947, reviews recent technological advances using unconventional soft materials, such as silicon/metal nanowires, functionalized hydrogels, and stretchable conductive nanocomposites. (Zhang, 2020), “Fully Organic Compliant Dry Electrodes Self-Adhesive to Skin for Long-Term Motion-Robust Epidermal Biopotential Monitoring,” Nature Communications, 2020, 11, 4683, reports an intrinsically conductive polymer dry electrode with excellent self-adhesiveness, stretchability, and conductivity.

Despite the above creative innovation and progress in this field, there still remains a need for better dry EEG electrode designs which can more-effectively penetrate between strands of hair to ensure good contact with a person's head without being uncomfortable or irritating the person's skin.

### SUMMARY OF THE INVENTION

This invention is a dry electrode (which does not use conductive gel) for use on a hair-covered area of a person's head. This electrode can be used to record EEG signals or deliver neurostimulation. This dry electrode has a plurality of electroconductive protrusions which extend outward from a base toward the surface of a person's head. These protrusions slide through the person's hair (e.g. slide between strands of hair), without being so narrow that they do not get good electrical contact and without being so stiff that they are uncomfortable for extended wear.

### INTRODUCTION TO THE FIGURES

FIG. 1 shows an electrode with a convex base and electroconductive protrusions.

FIG. 2 shows an electrode with a polygonal base and electroconductive protrusions.

FIG. 3 shows an electrode with a base and electroconductive protrusions, wherein the base is curved in a plane perpendicular to the surface of a person's head.

FIG. 4 shows an electrode with a flexible base and electroconductive protrusions, wherein the curvature of the base changes.

FIG. 5 shows an electrode with a flexible base and electroconductive protrusions, wherein tips of protrusions spread apart when the curvature of the base changes.

FIG. 6 shows an electrode with a base with moveable sections and electroconductive protrusions, wherein the moveable sections become more co-planar.

FIG. 7 shows an electrode with a base with moveable sections and electroconductive protrusions, wherein tips of protrusions spread apart when the moveable sections become more co-planar.

FIG. 8 shows an electrode with a base and electroconductive protrusions with arcuate cross sections.

FIG. 9 shows an electrode with a base and electroconductive protrusions with polygonal cross sections.

FIG. 10 shows an electrode with a base and tapered electroconductive protrusions.

FIG. 11 shows an electrode with a base and conic, frustal, or funnel shaped electroconductive protrusions

FIG. 12 shows an electrode with a base and conic-section-shaped electroconductive protrusions.

FIG. 13 shows an electrode with a base and paraboloidal-shaped electroconductive protrusions.

FIG. 14 shows an electrode with a base and electroconductive protrusions, wherein the cross section of a proximal section of a protrusion is larger than that of a distal section.

FIG. 15 shows an electrode with a base and electroconductive protrusions, wherein the cross section of a proximal section of a protrusion is smaller than that of a distal section.

FIG. 16 shows an electrode with a base and electroconductive protrusions, wherein there is a 90-degree angle between proximal and distal sections of a protrusion.

FIG. 17 shows an electrode with a base and electroconductive protrusions, wherein there is an obtuse angle between a proximal section of the protrusion and a distal section of the protrusion.

FIG. 18 shows an electrode with a base and electroconductive protrusions, wherein a protrusion has a columnar proximal section and a conic, frustal, or funnel shaped distal section.

FIG. 19 shows an electrode with a base and electroconductive protrusions, wherein a protrusion has a conic, frustal, and/or funnel shaped proximal section and a columnar distal section.

FIG. 20 shows an electrode with a base and electroconductive protrusions, wherein a protrusion has a columnar proximal section and a spheroidal distal section.

FIG. 21 shows an electrode with a base and electroconductive protrusions, wherein a protrusion has a conic, frustal, and/or funnel shaped proximal section and a spheroidal distal section.

FIG. 22 shows an electrode with a base and electroconductive protrusions, wherein a protrusion has a conic, frustal, and/or funnel shaped proximal and distal sections.

FIG. 23 shows an electrode with a base and electroconductive protrusions, wherein a protrusion has a conic, frustal, and/or funnel shaped proximal section and an inverted conic, frustal, and/or funnel shaped spheroidal distal section.

FIG. 24 shows an electrode with a base and electroconductive protrusions which are perpendicular to the base.

FIG. 25 shows an electrode with a base and electroconductive protrusions which extend out from the base at angles between 1 and 50 degrees.

FIG. 26 shows an electrode with a base and electroconductive protrusions which extend out from the base at angles between 40 and 60 degrees.

FIG. 27 shows an electrode with a base and electroconductive protrusions which extend out from the base at angles between 50 and 80 degrees.

FIG. 28 shows an electrode with a base and electroconductive protrusions which extend out from the base at angles between 70 and 89 degrees.

FIG. 29 shows an electrode with a base and concave electroconductive protrusions.

FIG. 30 shows an electrode with a base and convex electroconductive protrusions.

FIG. 31 shows an electrode with a base and articulated electroconductive protrusions, wherein the axis of a proximal section of a protrusion intersects the base at a greater angle than that of a distal section of the protrusion.

FIG. 32 shows an electrode with a base and articulated electroconductive protrusions, wherein the axis of a proximal section of a protrusion intersects the base at a lesser angle than that of a distal section of the protrusion.

FIG. 33 shows an electrode with a base and electroconductive protrusions, wherein a protrusion has a convex proximal section and a concave distal section.

FIG. 34 shows an electrode with a base and electroconductive protrusions, wherein a protrusion has a concave proximal section and a convex distal section.

FIG. 35 shows an electrode with a base and electroconductive protrusions which intersect radial spokes at angles between 1 and 45 degrees.

FIG. 36 shows an electrode with a base and electroconductive protrusions which intersect radial spokes at angles between 45 and 90 degrees.

FIG. 37 shows an electrode with a base and electroconductive protrusions which intersect radial spokes at angles between 90 and 135 degrees.

FIG. 38 shows an electrode with a base and electroconductive protrusions which intersect radial spokes at angles between 135 and 179 degrees.

FIG. 39 shows an electrode with a base and electroconductive protrusions, wherein the axis of a proximal section of a protrusion intersects the base at a first outward angle and intersects a radial spoke at first clockwise angle, wherein the axis of a distal section of a protrusion intersects the base at a second outward angle and intersects a radial spoke at second clockwise angle, wherein the second outward angle is less than the first outward angle, and wherein the second clockwise angle is greater than the first clockwise angle.

FIG. 40 shows an electrode with a base and electroconductive protrusions, wherein the axis of a proximal section of a protrusion intersects the base at a first outward angle and intersects a radial spoke at first clockwise angle, wherein the axis of a distal section of a protrusion intersects the base at a second outward angle and intersects a radial spoke at second clockwise angle, wherein the second outward angle is less than the first outward angle, and wherein the second clockwise angle is less than the first clockwise angle.

FIG. 41 shows an electrode with a base and electroconductive protrusions, wherein the axis of a proximal section of a protrusion intersects the base at a first outward angle and intersects a radial spoke at first clockwise angle, wherein the axis of a distal section of a protrusion intersects the base at a second outward angle and intersects a radial spoke at second clockwise angle, wherein the second outward angle is greater than the first outward angle, and wherein the second clockwise angle is greater than the first clockwise angle.

FIG. 42 shows an electrode with a base and electroconductive protrusions, wherein the axis of a proximal section of a protrusion intersects the base at a first outward angle and intersects a radial spoke at first clockwise angle, wherein the axis of a distal section of a protrusion intersects the base at a second outward angle and intersects a radial spoke at second clockwise angle, wherein the second outward angle is greater than the first outward angle, and wherein the second clockwise angle is less than the first clockwise angle.

FIG. 43 shows an electrode with a base and serpentine electroconductive protrusions.

FIG. 44 shows an electrode with a base and spiral and/or helical electroconductive protrusions.

FIG. 45 shows an electrode with a base and looping electroconductive protrusions.

FIG. 46 shows an electrode with a base and electroconductive protrusions which are each connected to the base at two or more points.

FIG. 47 shows an electrode with a base and semi-circular electroconductive protrusions.

FIG. 48 shows an electrode with a base and conic-section-shaped electroconductive protrusions.

FIG. 49 shows an electrode with a base and parabolic electroconductive protrusions.

FIG. 50 shows an electrode with a base and electroconductive protrusions with “U” and/or inverted croquet wicket shapes.

FIG. 51 shows an electrode with a base and electroconductive protrusions with “V” and/or a chevron shapes.

FIG. 52 shows an electrode with a base and bifurcating electroconductive protrusions.

FIG. 53 shows an electrode with a base and electroconductive protrusions, wherein proximal sections of protrusions are bifurcated.

FIG. 54 shows an electrode with a base and electroconductive protrusions, wherein distal sections of protrusions are bifurcated.

FIG. 55 shows an electrode with a base and electroconductive protrusions, wherein middle sections of protrusions are bifurcated.

FIG. 56 shows an electrode with a base and electroconductive protrusions, wherein protrusions farther from the center extend out at a greater outward-facing angle than protrusions closer to the center.

FIG. 57 shows an electrode with a base and electroconductive protrusions, wherein protrusions farther from the center extend out at a lesser outward-facing angle than protrusions closer to the center.

FIG. 58 shows an electrode with a base and electroconductive protrusions, wherein the angles between protrusions and the base vary with distance from the center.

FIG. 59 shows an electrode with a base and electroconductive protrusions, wherein the angles between protrusions and the base vary as a linear function of distance from the center.

FIG. 60 shows an electrode with a base and electroconductive protrusions, wherein the angles between protrusions and the base vary as a quadratic function of distance from the center.

FIG. 61 shows an electrode with a base and electroconductive protrusions, wherein protrusions closer to the center connect to the base at a lesser clockwise angle than protrusions closer to the center.

FIG. 62 shows an electrode with a base and electroconductive protrusions, wherein protrusions closer to the center connect to the base at a greater clockwise angle than protrusions closer to the center.

FIG. 63 shows an electrode with a base and electroconductive protrusions, wherein protrusions closer to the center have a smaller cross section.

FIG. 64 shows an electrode with a base and electroconductive protrusions, wherein protrusions closer to the center have a larger cross section.

FIG. 65 shows an electrode with a base and electroconductive protrusions, wherein protrusions closer to the center are shorter.

FIG. 66 shows an electrode with a base and electroconductive protrusions, wherein protrusions closer to the center are longer.

FIG. 67 shows an electrode with a base and electroconductive protrusions, wherein protrusions closer to the center are lower durometer.

FIG. 68 shows an electrode with a base and electroconductive protrusions, wherein protrusions closer to the center are higher durometer.

FIG. 69 shows an electrode with a base and electroconductive protrusions, wherein protrusions closer to the center are less elastic or flexible.

FIG. 70 shows an electrode with a base and electroconductive protrusions, wherein protrusions closer to the center are more elastic or flexible.

FIG. 71 shows an electrode with a base and electroconductive protrusions, wherein protrusions are evenly-distributed.

FIG. 72 shows an electrode with a base and electroconductive protrusions, wherein protrusions are around rings.

FIG. 73 shows an electrode with a base and electroconductive protrusions, wherein protrusions are along radial spokes.

FIG. 74 shows an electrode with a base and electroconductive protrusions, wherein protrusions are in a hub-and-spoke array.

FIG. 75 shows an electrode with a base and electroconductive protrusions, wherein protrusions are along spokes extending out from a midline.

FIG. 76 shows an electrode with a base and electroconductive protrusions, wherein protrusions are in a row-and-column array.

FIG. 77 shows an electrode with a base and electroconductive protrusions, wherein protrusions closer to the center are closer together.

FIG. 78 shows an electrode with a base and electroconductive protrusions, wherein protrusions closer to the center are farther apart.

FIG. 79 shows an electrode with a base and telescoping electroconductive protrusions.

FIG. 80 shows an electrode with a base and individually spring-loaded electroconductive protrusions.

FIG. 81 shows an electrode with a base and collectively spring-loaded electroconductive protrusions.

FIG. 82 shows an electrode with a base and electroconductive protrusions, wherein protrusions are individually pushed toward the surface of a person's head by compressible members.

FIG. 83 shows an electrode with a base and electroconductive protrusions, wherein protrusions are collectively pushed toward the surface of a person's head by a compressible member.

FIG. 84 shows an electrode with a base and electroconductive protrusions, wherein protrusions are individually pushed toward the surface of a person's head by pneumatic or hydraulic mechanisms.

FIG. 85 shows an electrode with a base and electroconductive protrusions, wherein protrusions are collectively pushed toward the surface of a person's head by a pneumatic or hydraulic mechanism.

FIG. 86 shows an electrode with a base and electroconductive protrusions, wherein protrusions are individually pushed toward the surface of a person's head by electromagnetic actuators.

FIG. 87 shows an electrode with a base and electroconductive protrusions, wherein protrusions are collectively pushed toward the surface of a person's head by an electromagnetic actuator.

FIG. 88 shows an electrode with a base and electroconductive protrusions, wherein individual protrusions are vibrated and/or oscillated by individual electromagnetic actuators.

FIG. 89 shows an electrode with a base and electroconductive protrusions, wherein protrusions are collectively vibrated and/or oscillated by an electromagnetic actuator.

FIG. 90 shows an electrode with a base and electroconductive protrusions, wherein pushing protrusions toward the surface of a person's head spreads their tips farther apart.

FIG. 91 shows an electrode with a base and electroconductive protrusions, wherein protrusions are shaped so that pushing them toward the surface of a person's head spreads their tips farther apart.

FIG. 92 shows an electrode with a base and articulated electroconductive protrusions, wherein pushing protrusions toward the surface of a person's head spreads their tips farther apart.

FIG. 93 shows an electrode with a base and electroconductive protrusions with hinges or joints, wherein pushing protrusions toward the surface of a person's head spreads their tips farther apart.

FIG. 94 shows an electrode with a base and electroconductive protrusions, wherein protrusions are individually vibrated and/or oscillated laterally by electromagnetic actuators.

FIG. 95 shows an electrode with a base and electroconductive protrusions, wherein protrusions are collectively vibrated and/or oscillated laterally by an electromagnetic actuator.

FIG. 96 shows an electrode with a base and electroconductive protrusions, wherein protrusions are individually rotated.

FIG. 97 shows an electrode with a base and electroconductive protrusions, wherein protrusions are collectively rotated.

FIG. 98 shows an electrode with a base and electroconductive protrusions, wherein the cross-sectional size of protrusions is increased after their insertion between strands of hair.

FIG. 99 shows an electrode with a base and electroconductive protrusions, wherein the cross-sectional size of protrusions is increased by electromagnetic actuators after their insertion between strands of hair.

FIG. 100 shows an electrode with a base and electroconductive protrusions, wherein the cross-sectional size of protrusions is increased by inserting material into their interiors after insertion of the protrusions between strands of hair.

FIG. 101 shows an electrode with a base and electroconductive protrusions, wherein the cross-sectional size of protrusions is increased by uncoiling them after inserting them between strands of hair.

FIG. 102 shows an electrode with a base and electroconductive protrusions, wherein the cross-sectional size of protrusions is increased by application of electrical energy after inserting the protrusions between strands of hair.

FIG. 103 shows an electrode with a base and electroconductive protrusions, wherein the durometer of protrusions is decreased after inserting them between strands of hair.

FIG. 104 shows an electrode with a base and electroconductive protrusions, wherein the flexibility or elasticity of protrusions is increased after inserting them between strands of hair.

### DETAILED DESCRIPTION OF THE FIGURES

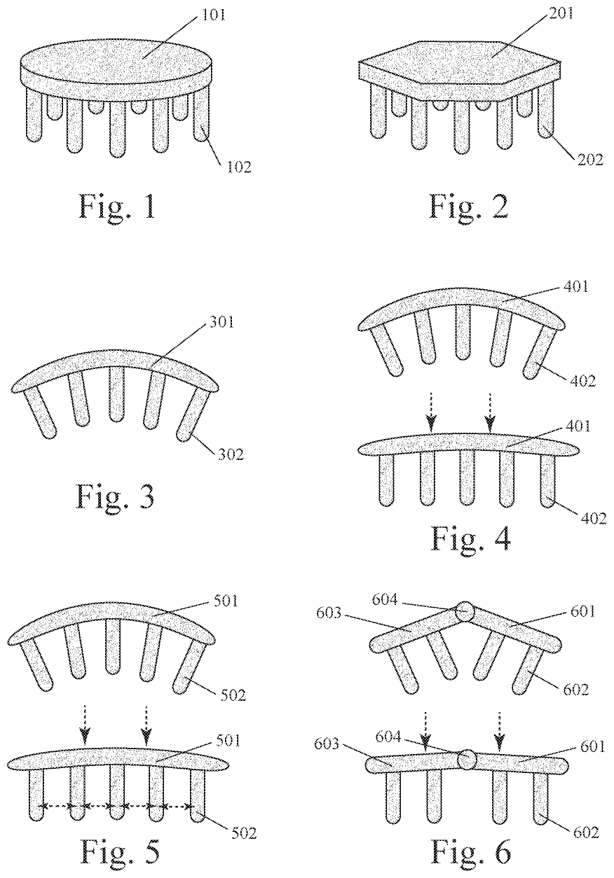

Before discussing the specific examples of dry EEG electrodes which are shown in FIGS. 1 through 104, it is useful to first discuss some general embodiment variations which can be applied later to these specific examples where relevant.

In an example, an electrode for use on a hair-covered area of a person's head can comprise: a base; and a plurality of electroconductive protrusions (e.g. protrusions, legs, teeth, prongs, pins, or loops) which are configured to extend out from base toward the surface of a person's head. In an example, electroconductive protrusions can extend through a person's hair to achieve good electrical and/or electromagnetic communication between the electrode and a hair-covered area of the person's head. In an example, electroconductive protrusions can be inserted and/or slide between strands of hair to achieve good electrical and/or electromagnetic communication between the electrode and a hair-covered area of a person's head.

In an example, a base can have a convex cross-sectional shape in a plane which is substantially parallel to the surface of a person's head. In an example, a base can have an arcuate (e.g. circular, elliptical, oval, or rounded polygonal) cross-sectional shape. In an example, a base can have a polygonal (e.g. square, rectangular, or hexagonal) cross-sectional shape. In an example, a base can be curved in a plane which is substantially perpendicular to the surface of a person's head. In an example, a base can be flexible and/or elastic. In an example, a flexible base can be changed between being concave, planar (e.g. flat), and convex relative to the surface of a person's head. In an example, a base can comprise a plurality of moveable sections which are connected by one or more hinges and/or moveable joints. In an example, these moveable sections can be changed between being coplanar and non-coplanar.

In an example, electroconductive protrusions can move (e.g. bend or slide) laterally (e.g. in a direction which is substantially parallel to the surface of the person's head) across the surface of a person's head while they are being pushed onto the surface of the person's head. In an example, electroconductive protrusions can slide laterally (e.g. in a direction which is substantially parallel to the surface of the person's head) across the surface of a person's head while they are pressed toward the surface of the person's head in order to slide between strands of the person's hair. This can achieve good electrical and/or electromagnetic communication between the electrode and a hair-covered area of the person's head.

In an example, a base can be flexible. In an example, a base can be changed from a first configuration which is convex relative to a person's head to a second configuration which is parallel (e.g. flat or planar) relative to the person's head. In an example, a base can be changed from a first configuration which is concave relative to a person's head to a second configuration which is parallel (e.g. flat or planar) relative to the person's head. In an example, the tips of electroconductive protrusions which protrude out from such a flexible base can move farther apart from each other as the base is changed from a first configuration to a second configuration. In an example, the tips of electroconductive protrusions can slide between strands of hair as they move farther apart from each other. This can achieve good electrical and/or electromagnetic communication between the electrode and a hair-covered area of the person's head.

In an example, the curvature of a base in a plane which is substantially perpendicular to the surface of a person's head can be changed from a first configuration to a second configuration when the base is pressed toward the surface of the person's head. In an example, the base can be less-curved (and more planar) in its second configuration than in its first configuration. In an example, the tips of the electroconductive protrusions can be moved farther apart to slide between strands of hair as a base is changed from its first configuration to its second configuration. This can achieve good electrical and/or electromagnetic communication between the electrode and a hair-covered area of the person's head.

In an example, a base can comprise two or more moveable sections. In an example, a base can comprise two or more moveable sections which are connected by one or more hinges or moveable joints. In an example, a base can have a first configuration and a second configuration, wherein moveable sections are more co-planar in the second configuration than in the first configuration. In an example, moveable sections of a base become more co-planar as the base is pressed toward the surface of a person's head. In an example, a base can be changed from the first configuration to the second configuration as the base is pressed toward the surface of the person's head. In an example, the tips of the electroconductive protrusions protruding from a base can be moved farther apart to slide between strands of hair as the base is changed from its first configuration to its second configuration.

In an example, electroconductive protrusions can be shaped so that they move (e.g. bend and/or slide) laterally (e.g. in a direction which is substantially parallel to the surface of the person's head) as they are pushed onto the surface of a person's head. In an example, this lateral movement can cause the tips of the protrusions to slide between strands of hair and achieve good electrical and/or electromagnetic communication between the electrode and the person's head.

In an example, electroconductive protrusions can be articulated, hinged, and/or jointed so that they move (e.g. bend and/or slide) laterally (e.g. in a direction which is substantially parallel to the surface of the person's head) as they are pushed onto the surface of the person's head. In an example, this lateral movement can cause the tips of the protrusions to slide between strands of hair and achieve good electrical and/or electromagnetic communication between the electrode and the person's head.

In an example, electroconductive protrusions can be moved (e.g. slid) laterally (e.g. in a direction which is substantially parallel to the surface of the person's head) by one or more electromagnetic actuators in order to achieve good electrical and/or electromagnetic communication between the electrode and the person's head. In an example, electroconductive protrusions can be moved (e.g. slid) laterally (e.g. in a direction which is substantially parallel to the surface of the person's head) by one or more electromagnetic actuators in order to slide between strands of the person's hair and enable good electrical and/or electromagnetic communication between the electrode and the person's head.

In an example, electroconductive protrusions can be vibrated and/or oscillated by one or more electromagnetic actuators. In an example, electroconductive protrusions can be vibrated and/or oscillated by one or more electromagnetic actuators in order to slide between strands of the person's hair and enable good electrical and/or electromagnetic communication between the electrode and the person's head.

In an example, electroconductive protrusions can be rotated by one or more electromagnetic actuators in order to achieve good electrical and/or electromagnetic communication between the electrode and the person's head. In an example, electroconductive protrusions can be rotated by one or more electromagnetic actuators in order to slide between strands of the person's hair and enable good electrical and/or electromagnetic communication between the electrode and the person's head.

In an example, electroconductive protrusions can have a first configuration during their insertion between strands of hair and a second configuration after their insertion between these stands of hair. In an example, protrusions can have a smaller cross-sectional size in their first configuration for easier insertion between strands of hair and a larger cross-sectional size in their second configuration for greater contact surface with the person's head and/or greater comfort for the person after their insertion between strands of hair. In an example, the cross-sectional size of a protrusion can be changed by a mechanism selected from the group consisting of: applying electrical energy to a protrusion made with material which expands upon application of electrical energy; inserting a longitudinal member (e.g. a rod or screw) into the center of a flexible protrusion; pumping a fluid or gas into a chamber inside a flexible protrusion; and uncoiling a protrusion using an electromagnetic actuator.

In an example, protrusions can be less flexible and/or elastic in their first configuration for easier insertion between strands of hair and a more flexible and/or elastic in their second configuration for greater contact surface with the person's head and/or greater comfort for the person. In an example, protrusions can have a higher durometer in their first configuration for easier insertion between strands of hair and a lower durometer in their second configuration for greater contact surface with the person's head and/or greater comfort for the person. In an example, the flexibility, elasticity, and/or durometer of a protrusion can be changed by application of electrical energy when the protrusion is made with material whose flexibility, elasticity, and/or durometer is changed by application of electrical energy.

In an example, electroconductive protrusions can have arcuate (e.g. circular, elliptical, oval, or rounded polygonal) cross-sectional shapes. In an example, electroconductive protrusions can have polygonal (e.g. square, rectangular, or hexagonal) cross-sectional shapes. In an example, an electroconductive protrusion can have a shape selected from the group consisting of: articulated, bifurcated, chevron, column, concave, cone, conic-section, convex, ellipsoid, frustum, funnel-shaped, helical, hourglass, loop, paraboloid, semicircle, serpentine, shaped like the letter “U”, shaped like the letter “V”, sinusoidal, spheroid, spiral, and tapered.

In an example, an electroconductive protrusion can have a compound shape. In an example, an electroconductive protrusion can comprise a proximal section which is closer to the base and a distal section which is father from the base. In an example, a proximal section of an electroconductive protrusion can comprise the proximal 50% (e.g. half) of the length (e.g. longitudinal axis) of an electroconductive protrusion and the distal section of the electroconductive protrusion can comprise the distal 50% (e.g. half) of the electroconductive protrusion. In an example, a proximal section of an electroconductive protrusion can comprise between 25% and 50% of the length (e.g. longitudinal axis) of an electroconductive protrusion and the distal section of the electroconductive protrusion can comprise the remaining length of the electroconductive protrusion. In an example, a proximal section of an electroconductive protrusion can comprise between 50% and 75% of the length (e.g. longitudinal axis) of an electroconductive protrusion and the distal section of the electroconductive protrusion can comprise the remaining length of the electroconductive protrusion.

In an example, proximal and distal sections of an electroconductive protrusion can intersect each other at an acute angle. In an example, proximal and distal sections of an electroconductive protrusion can intersect each other at an obtuse angle. In an example, proximal and distal sections of an electroconductive protrusion can intersect each other at a 45-degree angle. In an example, proximal and distal sections of an electroconductive protrusion can intersect each other at a 90-degree angle. In an example, proximal and distal sections of an electroconductive protrusion can intersect each other at an angle in the range (including end points) of 20 to 45 degrees. In an example, proximal and distal sections of an electroconductive protrusion can intersect each other at an angle in the range (including end points) of 40 to 85 degrees. In an example, proximal and distal sections of an electroconductive protrusion can intersect each other at an angle in the range (including end points) of 85 to 135 degrees.

In an example, central longitudinal axes of proximal and distal sections of an electroconductive protrusion can intersect each other at an acute angle. In an example, central longitudinal axes of proximal and distal sections of an electroconductive protrusion can intersect each other at an obtuse angle. In an example, central longitudinal axes of proximal and distal sections of an electroconductive protrusion can intersect each other at a 45-degree angle. In an example, central longitudinal axes of proximal and distal sections of an electroconductive protrusion can intersect each other at a 90-degree angle. In an example, central longitudinal axes of proximal and distal sections of an electroconductive protrusion can intersect each other at an angle in the range (including end points) of 20 to 45 degrees. In an example, central longitudinal axes of proximal and distal sections of an electroconductive protrusion can intersect each other at an angle in the range (including end points) of 40 to 85 degrees. In an example, central longitudinal axes of proximal and distal sections of an electroconductive protrusion can intersect each other at an angle in the range (including end points) of 85 to 135 degrees.

In an example, a distal section of an electroconductive protrusion can be narrower (e.g. have a smaller average cross-sectional size) than a proximal section of the electroconductive protrusion. In an example, a distal section of an electroconductive protrusion can be wider (e.g. have a larger average cross-sectional size) than a proximal section of the electroconductive protrusion. In an example, the maximum width of a distal section of an electroconductive protrusion can be at least 50% wider than the maximum width of a proximal section of the electroconductive protrusion. In an example, the maximum width of a proximal section of an electroconductive protrusion can be at least 50% wider than the maximum width of a distal section of the electroconductive protrusion.

In an example, a proximal section of an electroconductive protrusion can have a shape selected from the group consisting of: articulated, bifurcated, chevron, column, concave, cone, conic-section, convex, ellipsoid, frustum, funnel-shaped, helical, hourglass, loop, paraboloid, semicircle, serpentine, shaped like the letter “U”, shaped like the letter “V”, sinusoidal, spheroid, spiral, and tapered. In an example, a distal section of an electroconductive protrusion can have a shape selected from the group consisting of: articulated, bifurcated, chevron, column, concave, cone, conic-section, convex, ellipsoid, frustum, funnel-shaped, helical, hourglass, loop, paraboloid, semicircle, serpentine, shaped like the letter “U”, shaped like the letter “V”, sinusoidal, spheroid, spiral, and tapered.

In an example, a protrusion can comprise: a proximal section with a first shape which is selected from the group consisting of: articulated, bifurcated, chevron, column, concave, cone, conic-section, convex, ellipsoid, frustum, funnel-shaped, helical, hourglass, loop, paraboloid, semicircle, serpentine, shaped like the letter “U”, shaped like the letter “V”, sinusoidal, spheroid, spiral, and tapered; and a distal section with a second shape selected from the group consisting of: articulated, bifurcated, chevron, column, concave, cone, conic-section, convex, ellipsoid, frustum, funnel-shaped, helical, hourglass, loop, paraboloid, semicircle, serpentine, shaped like the letter “U”, shaped like the letter “V”, sinusoidal, spheroid, spiral, and tapered.

In an example, a distal section of a protrusion can be more conductive than the proximal section of the protrusion. In an example, a distal section of a protrusion can be more flexible and/or elastic than the proximal section of the protrusion. In an example, a distal section of a protrusion can be more have a lower durometer level than the proximal section of the protrusion. In an example, the core of a protrusion can be less conductive than an outer layer of a protrusion. In an example, the core of a protrusion can be more conductive than an outer layer of a protrusion. In an example, the core of a protrusion can have a higher durometer level than that of an outer layer of a protrusion. In an example, the core of a protrusion can have a lower durometer level than that of an outer layer of a protrusion.

In an example, different electroconductive protrusions can extend out from a base at different outward-facing angles. In an example, electroconductive protrusions which are closer to the center of a base can extend out from a base at greater outward-facing angles than electroconductive protrusions which are farther from the center. In an example, electroconductive protrusions which are closer to the center of a base can extend out from a base at lesser outward-facing angles than electroconductive protrusions which are farther from the center. In an example, electroconductive protrusions can extend out from a base at outward-facing angles which vary as a function of distance from the center of the base. In an example, electroconductive protrusions can extend out from a base at outward-facing angles which vary as a linear function of distance from the center of the base. In an example, electroconductive protrusions can extend out from a base at outward-facing angles which vary as a quadratic function of distance from the center of the base.

In an example, electroconductive protrusions can extend out from a base along vectors which are parallel to virtual radial spokes which extend out from the center of a base. In an example, electroconductive protrusions can extend out along vectors which intersect virtual radial spokes at non-zero clockwise-facing angles. In an example, electroconductive protrusions can extend out along vectors which intersect virtual radial spokes at clockwise-facing angles in the range (including end points) of 1 to 45 degrees. In an example, electroconductive protrusions can extend out along vectors which intersect virtual radial spokes at clockwise-facing angles in the range (including end points) of 45 to 90 degrees. In an example, electroconductive protrusions can extend out along vectors which intersect virtual radial spokes at clockwise-facing angles in the range (including end points) of 90 to 135 degrees. In an example, electroconductive protrusions can extend out along vectors which intersect virtual radial spokes at clockwise-facing angles in the range (including end points) of 135 to 179 degrees.

In an example, electroconductive protrusions can be evenly distributed (e.g. evenly-spaced) across the head-facing surface of a base. In an example, electroconductive protrusions can be distributed in a pattern of nested (e.g. concentric) rings on a base. In an example, electroconductive protrusions can be distributed along radial spokes extending out from the center of a base. In an example, electroconductive protrusions can be distributed in a hub-and-spoke configuration on a base. In an example, electroconductive protrusions can be distributed in a row-and-column array (e.g. array, matrix, or grid) on a base. In an example, electroconductive protrusions can be distributed in a honeycomb array (e.g. hexagonal grid) on a base. In an example, electroconductive protrusions can be distributed in a spiral pattern on a base. In an example, electroconductive protrusions can be distributed along spokes extending out from a midline of a base.

In an example, electroconductive protrusions which are closer to the center (or midline) of a base can be closer together than electroconductive protrusions which are father from the center (or midline) of the base. In an example, electroconductive protrusions which are closer to the center (or midline) of a base can be farther apart than electroconductive protrusions which are father from the center (or midline) of the base. In an example, electroconductive protrusions around rings which are closer to the center (or midline) of a base can be closer together than electroconductive protrusions around rings which are father from the center (or midline) of the base. In an example, electroconductive protrusions around rings which are closer to the center (or midline) of a base can be farther apart than electroconductive protrusions around rings which are father from the center (or midline) of the base.

In an example, electroconductive protrusions which are closer to the center (or midline) of a base can be wider than electroconductive protrusions which are farther from the center (or midline). In an example, electroconductive protrusions which are closer to the center (or midline) of a base can be narrower than electroconductive protrusions which are farther from the center (or midline). In an example, electroconductive protrusions which are closer to the center (or midline) of a base can be longer than electroconductive protrusions which are farther from the center (or midline). In an example, electroconductive protrusions which are closer to the center (or midline) of a base can be shorter than electroconductive protrusions which are farther from the center (or midline).

In an example, electroconductive protrusions which are closer to the center (or midline) of a base can have a lower durometer level than electroconductive protrusions which are farther from the center (or midline). In an example, electroconductive protrusions which are closer to the center (or midline) of a base can have a higher durometer level than electroconductive protrusions which are farther from the center (or midline). In an example, electroconductive protrusions which are closer to the center (or midline) of a base can be more flexible or elastic than electroconductive protrusions which are farther from the center (or midline). In an example, electroconductive protrusions which are closer to the center (or midline) of a base can be less flexible or elastic than electroconductive protrusions which are farther from the center (or midline).

In an example, electroconductive protrusions which are closer to the center (or midline) of a base can each have a first shape selected from the group consisting of: articulated, bifurcated, chevron, column, concave, cone, conic-section, convex, ellipsoid, frustum, funnel-shaped, helical, hourglass, loop, paraboloid, semicircle, serpentine, shaped like the letter “U”, shaped like the letter “V”, sinusoidal, spheroid, spiral, and tapered; and electroconductive protrusions which are farther from the center (or midline) or a base can each have a second shape selected from the group consisting of: articulated, bifurcated, chevron, column, concave, cone, conic-section, convex, ellipsoid, frustum, funnel-shaped, helical, hourglass, loop, paraboloid, semicircle, serpentine, shaped like the letter “U”, shaped like the letter “V”, sinusoidal, spheroid, spiral, and tapered.

In an example, an electrode configured for use on a hair-covered area of a person's head can comprise: a base, wherein the base has a first configuration with a first amount of curvature, wherein the base has a second configuration with a second amount of curvature, wherein the second amount is less than the first amount, and wherein the base is changed from the first configuration to the second configuration when the base is pressed onto a person's head; and electroconductive protrusions which extend out from the base, wherein there is a first average distance between tips of the protrusions in the first configuration, wherein there is a second average distance between tips of the protrusions in the second configuration, and wherein the second average distance is greater than the first average distance. In an example, the base can comprise a continuous flexible structure. In an example, the base can comprise a plurality of connected moveable sections. In an example, moveable sections can be closer to being co-planar in the second configuration than in the first configuration.

In an example, an electrode configured for use on a hair-covered area of a person's head can comprise: a base; and electroconductive protrusions which extend out from the base, wherein the protrusions are configured so that pushing the protrusions toward the surface of the person's head causes tips of the protrusions to move farther apart from each other. In an example, the protrusions can be articulated so that pushing the protrusions toward the surface of the person's head causes tips of the protrusions to move farther apart from each other. In an example, the protrusions can be articulated with hinges or movable joints so that pushing the protrusions toward the surface of the person's head causes tips of the protrusions to move farther apart from each other.

In an example, a protrusion can have a proximal section which is closer to the base and a distal section which is farther from the base, wherein the proximal section has a central longitudinal axis which intersects the plane of the base at a first outward-facing angle, and wherein the distal section has a central longitudinal axis whose virtual extension intersects the plane of the base at a second outward-facing angle. In an example, the second outward-facing angle can be less than the first outward-facing angle. In an example, the second outward-facing angle can be greater than the first outward-facing angle. In an example, protrusions which are closer to the center of the base can connect to the base at a first average outward-facing angle, wherein protrusions which are farther from the center of the base can connect to the base at a second average outward-facing angle, and wherein the second average outward-facing angle is less than the first average outward-facing angle.

In an example, a protrusion can be concave relative to a central longitudinal axis of the protrusion. In an example, a protrusion can be convex relative to a central longitudinal axis of the protrusion. In an example, a protrusion can have a proximal section which is closer to the base and a distal section which is farther from the base, wherein the proximal section is convex relative to a central longitudinal axis of the protrusion, and wherein the distal section is concave relative to a central longitudinal axis of the protrusion. In an example, a protrusion can have a proximal section which is closer to the base and a distal section which is farther from the base, wherein the proximal section is concave relative to a central longitudinal axis of the protrusion, and wherein the distal section is convex relative to a central longitudinal axis of the protrusion.

In an example, a protrusion can have a proximal section which is closer to the base and a distal section which is farther from the base, and wherein the proximal section is bifurcated. In an example, a protrusion can have a proximal section which is closer to the base and a distal section which is farther from the base, and wherein the distal section is bifurcated. In an example, a protrusion can have a proximal section which is closer to the base, a distal section which is farther from the base, and a middle section between the proximal section and the distal section; and wherein the middle section is bifurcated.

In an example, an electrode configured for use on a hair-covered area of a person's head can comprise: a base; and electroconductive protrusions which extend out from the base, wherein the protrusions have a first configuration with a first average cross-sectional size, wherein the protrusions have a second configuration with a second cross-sectional size, wherein the second cross-sectional size is greater than the first cross-sectional size, and wherein the protrusions are changed from the first configuration to the second configuration after the protrusions have been inserted between strands of hair on the surface of a person's head. In an example, protrusions can be expanded from the first configuration to the second configuration by an expansion mechanism selected from the group consisting of: application of electrical energy to the protrusions; inserting solid matter into the interior of the protrusions; pumping a liquid or gas into the interior of the protrusions; and uncoiling the protrusions.

In an example, an electrode configured for use on a hair-covered area of a person's head can comprise: a base, wherein the base has a first configuration with a first amount of curvature, wherein the base has a second configuration with a second amount of curvature, wherein the second amount is less than the first amount, and wherein the base is changed from the first configuration to the second configuration when the base is pressed onto a person's head; and electroconductive protrusions which extend out from the base, wherein there is a first average distance between tips of the protrusions in the first configuration, wherein there is a second average distance between tips of the protrusions in the second configuration, and wherein the second average distance is greater than the first average distance.

In an example, a base can be flexible. In an example, a base can comprise a continuous flexible structure. In an example, a base can comprise a single flexible structure. In an example, a base can comprise a flexible structure which deforms in response to pressure. In an example, a base can comprise a flexible structure which can be flattened by pressure from a head-worn EEG device. In an example, a base can have a concave shape when not subjected to external force and can have a substantially-flat shape when subjected to external force. In an example, a base can have a concave shape before being pressed onto a person's head and can have a substantially-flat shape after being pressed onto the person's head.

In an example, a base can comprise a plurality of connected moveable sections. In an example, moveable sections which collectively comprise a base can have individual shapes selected from the group consisting of: square; rectangular; hexagonal; trapezoidal; and semicircular. In an example, a base can comprise a plurality of moveable sections which are connected by hinges or moveable joints. In an example, a base can comprise a plurality of flat moveable sections which are connected by hinges or moveable joints. In an example, moveable sections comprising a base can collectively form a convex structure in a first configuration and a substantially-flat structure in a second configuration. In an example, moveable sections can be closer to being co-planar in the second configuration than in the first configuration.

In an example, a base can be flatter in the second configuration than in the first configuration. In an example, a base can be more planar (e.g. closer to fitting within a flat plane) in the second configuration than in the first configuration. In an example, the amount of curvature of a base can be relative to the surface of a person's head. In an example, the amount of curvature of a base can be measured in degrees. In an example, a base can be concave before the electrode has been pressed against the surface of a person's head and can be substantially flat after the electrode has been pressed onto the surface of a person's head. In an example, a base can have a first degree of concavity before the electrode has been pressed against the surface of a person's head and a second degree of concavity after the electrode has been pressed onto the surface of a person's head, wherein the second degree is less than the first degree.

In an example, protrusions can be non-parallel to each other before the electrode has been pressed against the surface of a person's head and can be substantially-parallel to each other after the electrode has been pressed onto the surface of a person's head. In an example, pushing an electrode onto the surface of a person's head flattens the base and spreads the tips of protrusions farther apart, thereby sliding the tips between strands of hair on the person's head. In an example, when a base is curved, then protrusions can extend out from a base in a manner which is orthogonal (e.g. perpendicular) to the local curvature of the base at the point of connection between the protrusion and the base. In an example, when a base is not curved (e.g. flat), then protrusions can extend out from the base in a manner which is orthogonal (e.g. perpendicular) to the flat plane of the base.

In an example, the tips of protrusions can be pushed radially outward, away from the central axis of a base, when an electrode is pushed onto the surface of a person's head. In an example, pushing these tips radially outward causes them to move across the surface of the person's head and thereby slide through hair (e.g. slide between strands of hair). In an example, protrusions can be tilted away from the central axis of a base when an electrode is pushed onto the surface of a person's head. In an example, protrusions can be tilted into configurations which are more perpendicular to the surface of a person's head when the electrode is pushed onto the surface of a person's head. In an example, tilting the protrusions causes their tips to move across the surface of the person's head and thereby slide through hair (e.g. slide between strands of hair).

In an example, an electrode configured for use on a hair-covered area of a person's head can comprise: a base; and electroconductive protrusions which extend out from the base, wherein the protrusions are configured so that pushing the protrusions toward the surface of the person's head causes tips of the protrusions to move farther apart from each other. In an example, the protrusions can be articulated so that pushing the protrusions toward the surface of the person's head causes tips of the protrusions to move farther apart from each other. In an example, the protrusions can be articulated with hinges or movable joints so that pushing the protrusions toward the surface of the person's head causes tips of the protrusions to move farther apart from each other.

In an example, protrusions can comprise two or more sections which are attached to each other by movable joints like a pair of scissors. In an example, protrusions can comprise two or more articulated sections which are attached to each other by movable joints, thereby enabling a scissor movement. In an example, protrusions can comprise two or more sections which are attached to each other at their mid-sections (e.g. between proximal and distal portions) by movable joints or hinges. In an example, protrusions can comprise two or more sections which are attached to each other at their distal ends (e.g. farthest from the base) by movable joints or hinges.

In an example, a protrusion can comprise two or more sections which are attached to each other by movable joints like a pair of scissors. In an example, a protrusion can comprise two or more articulated sections which are attached to each other by movable joints, thereby enabling a scissor movement. In an example, a protrusion can comprise two of more sections which are attached to each other at their mid-sections (e.g. between proximal and distal portions) by movable joints or hinges. In an example, a protrusion can comprise two or more sections which are attached to each other at their distal ends (e.g. farthest from the base) by movable joints or hinges.

In an example, the angle between proximal and distal sections of a protrusion can change as an electrode is pressed onto the surface of a person's head, thereby causing the tip of the protrusion to slide laterally over the surface of the person's head. In an example, the outward-facing angle between proximal and distal sections of a protrusion can decrease as an electrode is pressed onto the surface of a person's head, thereby causing the tip of the protrusion to slide laterally over the surface of the person's head. In an example, the outward-facing angle between proximal and distal sections of a protrusion can increase as an electrode is pressed onto the surface of a person's head, thereby causing the tip of the protrusion to slide laterally over the surface of the person's head. In an example, the angle between proximal and distal sections of a protrusion can change in a scissor-like manner as an electrode is pressed onto the surface of a person's head, thereby causing the tip of the protrusion to slide laterally over the surface of the person's head.

In an example, a protrusion can be articulated so that longitudinal (e.g. toward the surface of the person's head) movement of the protrusion also causes lateral (e.g. across the surface of the person's head) movement of the tip of the protrusion. In an example, a protrusion can be articulated with multiple jointed sections so that longitudinal (e.g. toward the surface of the person's head) movement of the protrusion also causes lateral (e.g. across the surface of the person's head) movement of the tip of the protrusion. In an example, a protrusion can be articulated like a pair of scissors so that longitudinal (e.g. toward the surface of the person's head) movement of the protrusion also causes lateral (e.g. across the surface of the person's head) movement of the tip of the protrusion.

In an example, a protrusion can have a proximal section which is closer to the base and a distal section which is farther from the base, wherein the proximal section has a central longitudinal axis which intersects the plane of the base at a first outward-facing angle, and wherein the distal section has a central longitudinal axis whose virtual extension intersects the plane of the base at a second outward-facing angle. In an example, the second outward-facing angle can be less than the first outward-facing angle. In an example, the second outward-facing angle can be greater than the first outward-facing angle. In an example, protrusions which are closer to the center of the base can connect to the base at a first average outward-facing angle, wherein protrusions which are farther from the center of the base can connect to the base at a second average outward-facing angle, and wherein the second average outward-facing angle is less than the first average outward-facing angle.

In an example, a protrusion can be concave relative to a central longitudinal axis of the protrusion. In an example, a protrusion can be convex relative to a central longitudinal axis of the protrusion. In an example, a protrusion can have a proximal section which is closer to the base and a distal section which is farther from the base, wherein the proximal section is convex relative to a central longitudinal axis of the protrusion, and wherein the distal section is concave relative to a central longitudinal axis of the protrusion. In an example, a protrusion can have a proximal section which is closer to the base and a distal section which is farther from the base, wherein the proximal section is concave relative to a central longitudinal axis of the protrusion, and wherein the distal section is convex relative to a central longitudinal axis of the protrusion.

In an example, a protrusion can have a proximal section which is closer to the base and a distal section which is farther from the base, and wherein the proximal section is bifurcated. In an example, a protrusion can have a proximal section which is closer to the base and a distal section which is farther from the base, and wherein the distal section is bifurcated. In an example, a protrusion can have a proximal section which is closer to the base, a distal section which is farther from the base, and a middle section between the proximal section and the distal section; and wherein the middle section is bifurcated.

In an example, an electrode configured for use on a hair-covered area of a person's head can comprise: a base; and electroconductive protrusions which extend out from the base, wherein the protrusions have a first configuration with a first average cross-sectional size, wherein the protrusions have a second configuration with a second cross-sectional size, wherein the second cross-sectional size is greater than the first cross-sectional size, and wherein the protrusions are changed from the first configuration to the second configuration after the protrusions have been inserted between strands of hair on the surface of a person's head.

In an example, protrusions can be expanded from the first configuration to the second configuration by an expansion mechanism selected from the group consisting of: application of electrical energy to the protrusions; inserting solid matter into the interior of the protrusions; pumping a liquid or gas into the interior of the protrusions; and uncoiling the protrusions. In an example, a protrusion can be made from an electroactive polymer which expands when stimulated by electrical energy and/or an electric field. In an example, a protrusion can be cross-sectionally expanded by inserting a rod into the core of the protrusion. In an example, a protrusion can be cross-sectionally expanded by pumping a liquid or gas into a chamber within the protrusion. In an example, a protrusion can be longitudinally coiled or uncoiled, wherein uncoiling the protrusion causes it to cross-sectionally expand.

In an example, a protrusion can be radially-asymmetric. In an example, a cross-section of a protrusion can be radially-asymmetric. This cross-section is perpendicular to a longitudinal axis of the protrusion. In an example: a first portion (e.g. half or side) of a cross-section of a protrusion can be made with a first material and a second portion (e.g. half or side) of the cross-section of the protrusion can be made with a second material, wherein the first material is more flexible, more elastic, and/or have a lower durometer than the second material, thereby biasing the protrusion to bend in a selected lateral direction (and slide between strands of hair) when the protrusion is pressed onto the surface of a person's head. In an example, this selected lateral direction can be radially outward and away from the center of the base. In an example, this selected lateral direction can be away from (a virtual extension of) the central longitudinal axis of the base.

In an example: a first portion (e.g. half or side) of a cross-section of a protrusion which faces away from the center of a base can be made with a first material and a second portion (e.g. half or side) of the cross-section of the protrusion which faces toward the center of the base can be made with a second material, wherein the first material is more flexible, be more elastic, and/or have a lower durometer than the second material, thereby biasing the protrusion to bend laterally away from the center of the base in a selected lateral direction (and slide between strands of hair) when the protrusion is pressed onto the surface of a person's head.

In an example: a first portion (e.g. half or side) of a cross-section of a protrusion which faces away from (a virtual extension of) the central longitudinal axis of the base can be made with a first material and a second portion (e.g. half or side) of the cross-section of the protrusion which faces toward (a virtual extension of) the central longitudinal axis of the base can be made with a second material, wherein the first material is more flexible, be more elastic, and/or have a lower durometer than the second material, thereby biasing the protrusion to bend laterally away from (a virtual extension of) the central longitudinal axis of the base in a selected lateral direction (and slide between strands of hair) when the protrusion is pressed onto the surface of a person's head.

In an example: a first portion (e.g. half or side) of a cross-section of a protrusion can comprise a first spring or coil and a second portion (e.g. half or side) of the cross-section of the protrusion can comprise a second spring or coil, wherein the first spring or coil is less resistant and/or more compressible than the second spring or coil, thereby biasing the protrusion to bend in a selected lateral direction (and slide between strands of hair) when the protrusion is pressed onto the surface of a person's head.

In an example: a first portion (e.g. half or side) of a cross-section of a protrusion can comprise a first elastic band and a second portion (e.g. half or side) of the cross-section of the protrusion can comprise a second elastic band, wherein the first elastic band is more elastic and/or stretchable than the second elastic band, thereby biasing the protrusion to bend in a selected lateral direction (and slide between strands of hair) when the protrusion is pressed onto the surface of a person's head.

In an example: a first portion (e.g. half or side) of a cross-section of a protrusion can comprise a first rod, strap, or column and a second portion (e.g. half or side) of the cross-section of the protrusion can comprise a second rod, strap, or column, wherein the first rod, strap, or column moves and/or shifts relative to the second rod, strap, or column when the protrusion is pressed onto the surface of a person's head, thereby biasing the protrusion to bend in a selected lateral direction and slide between strands of hair.

In an example: a first portion (e.g. half or side) of a cross-section of a protrusion can comprise a first rod, strap, or column and a second portion (e.g. half or side) of the cross-section of the protrusion can comprise a second rod, strap, or column, wherein the first rod, strap, or column is more flexible, be more elastic, and/or have a lower durometer than the second rod, strap, or column, thereby biasing the protrusion to bend in a selected lateral direction (and slide between strands of hair) when the protrusion is pressed onto the surface of a person's head.

This disclosure now discusses the example embodiments of this invention which are shown in FIGS. 1 through 104. Relevant variations and claim limiting specifications which were discussed in the preceding section can be applied to the examples shown in these figures.

FIG. 1 shows an electrode with a convex base and electroconductive protrusions. More specifically, FIG. 1 shows an example of an oblique side view of a dry electrode for use on a hair-covered area comprising: a base 101; and electroconductive protrusions, including 102, which extend out from the base; wherein the base has a convex (e.g. circular, ellipsoidal, or oval) shape in a plane which is substantially parallel to the surface of a person's head. Relevant variations and claim limiting specifications discussed elsewhere in this disclosure or in priority-claimed disclosures can also be added to the example shown in this figure.

FIG. 2 shows an electrode with a polygonal base and electroconductive protrusions. More specifically, FIG. 2 shows an example of an oblique side view of a dry electrode for use on a hair-covered area comprising: a base 201; and electroconductive protrusions, including 202, which extend out from the base; wherein the base has a polygonal (e.g. square, rectangular, or hexagonal) shape in a plane which is substantially parallel to the surface of a person's head. Relevant variations and claim limiting specifications discussed elsewhere in this disclosure or in priority-claimed disclosures can also be added to the example shown in this figure.

FIG. 3 shows an electrode with a base and electroconductive protrusions, wherein the base is curved in a plane perpendicular to the surface of a person's head. More specifically, FIG. 3 shows an example of a side view of a dry electrode for use on a hair-covered area comprising: a base 301; and electroconductive protrusions, including 302, which extend out from the base; wherein the base has an arcuate (e.g. concave or convex) shape in a plane which is substantially perpendicular to the surface of a person's head. Relevant variations and claim limiting specifications discussed elsewhere in this disclosure or in priority-claimed disclosures can also be added to the example shown in this figure.

FIG. 4 shows an electrode with a flexible base and electroconductive protrusions, wherein the curvature of the base changes. More specifically, FIG. 4 shows an example of two sequential side views of a dry electrode for use on a hair-covered area comprising: a flexible base 401; and electroconductive protrusions, including 402, which extend out from the base; wherein the base has a first configuration with a first degree of curvature relative to the surface of a person's head, wherein the base has a second configuration with a second degree of curvature relative to the surface of a person's head, wherein the second degree is less than the first degree, and wherein the base is changed from the first configuration to the second configuration when the base is pressed onto a person's head. Relevant variations and claim limiting specifications discussed elsewhere in this disclosure or in priority-claimed disclosures can also be added to the example shown in this figure.

FIG. 5 shows an electrode with a flexible base and electroconductive protrusions, wherein tips of protrusions spread apart when the curvature of the base changes. More specifically, FIG. 5 shows an example of two sequential side views of a dry electrode for use on a hair-covered area comprising: a flexible base 501; and electroconductive protrusions, including 502, which extend out from the base; wherein the base has a first configuration with a first degree of curvature relative to the surface of a person's head, wherein the base has a second configuration with a second degree of curvature relative to the surface of a person's head, wherein the second degree is less than the first degree, wherein the base is changed from the first configuration to the second configuration when the base is pressed onto a person's head, wherein there is a first average distance between the tips of the electroconductive protrusions in the first configuration, wherein there is a second average distance between tips of the electroconductive protrusions in the second configuration, and wherein the second average distance is greater than the first average distance. Relevant variations and claim limiting specifications discussed elsewhere in this disclosure or in priority-claimed disclosures can also be added to the example shown in this figure.

FIG. 6 shows an electrode with a base with moveable sections and electroconductive protrusions, wherein the moveable sections become more co-planar. More specifically, FIG. 6 shows an example of two sequential side views of a dry electrode for use on a hair-covered area comprising: a base 601; and electroconductive protrusions, including 602, which extend out from the base; wherein the base further comprises a plurality of moveable sections which are connected to each other by hinges and/or joints, wherein the base has a first configuration before it is pressed against a person's head, wherein the base has a second configuration after it is pressed against the person's head, and wherein the plurality of moveable sections are closer to being co-planar in the second configuration than in the first configuration. Relevant variations and claim limiting specifications discussed elsewhere in this disclosure or in priority-claimed disclosures can also be added to the example shown in this figure.

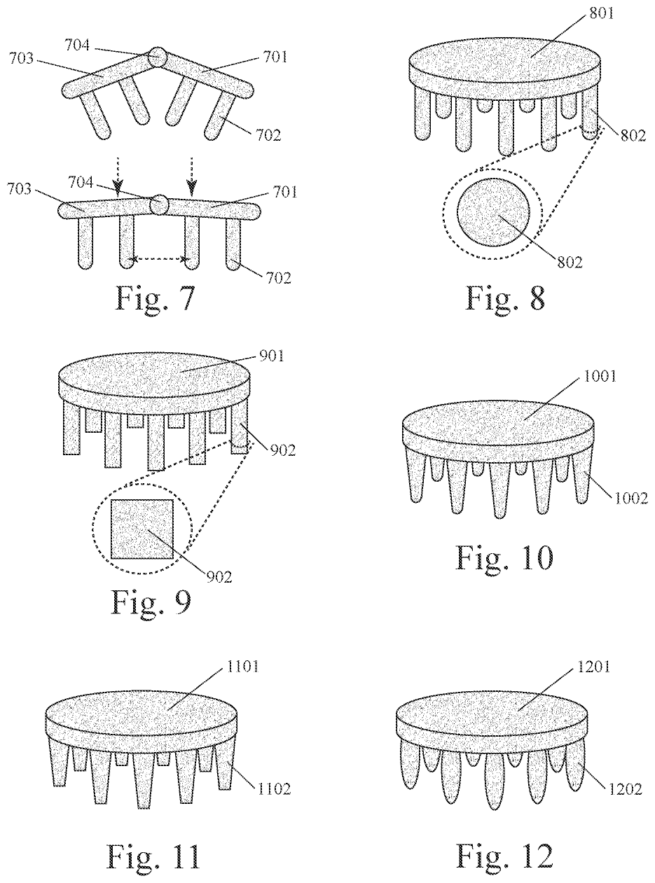

FIG. 7 shows an electrode with a base with moveable sections and electroconductive protrusions, wherein tips of protrusions spread apart when the moveable sections become more co-planar. More specifically, FIG. 7 shows an example of two sequential side views of a dry electrode for use on a hair-covered area comprising: a base 701; and electroconductive protrusions, including 702, which extend out from the base; wherein the base further comprises a plurality of moveable sections which are connected to each other by hinges and/or joints, wherein the base has a first configuration before it is pressed against a person's head, wherein the base has a second configuration after it is pressed against the person's head, wherein the plurality of moveable sections are closer to being co-planar in the second configuration than in the first configuration, wherein there is a first average distance between tips of the electroconductive protrusions in the first configuration, wherein there is a second average distance between the tips of the electroconductive protrusions in the second configuration, and wherein the second average distance is greater than the first average distance. Relevant variations and claim limiting specifications discussed elsewhere in this disclosure or in priority-claimed disclosures can also be added to the example shown in this figure.

FIG. 8 shows an electrode with a base and electroconductive protrusions with arcuate cross sections. More specifically, FIG. 8 shows an example of an oblique side view of a dry electrode for use on a hair-covered area comprising: a base 801; and electroconductive protrusions, including 802, which extend out from the base, wherein protrusions have arcuate (e.g. circular, ellipsoidal, oval, or tear-drop) cross-sectional shapes [shown in dotted-line close-up sub-view]. Relevant variations and claim limiting specifications discussed elsewhere in this disclosure or in priority-claimed disclosures can also be added to the example shown in this figure.

FIG. 9 shows an electrode with a base and electroconductive protrusions with polygonal cross sections. More specifically, FIG. 9 shows an example of an oblique side view of a dry electrode for use on a hair-covered area comprising: a base 901; and electroconductive protrusions, including 902, which extend out from the base, wherein protrusions have polygonal (e.g. square, rectangular, or hexagonal) cross-sectional shapes [shown in dotted-line close-up sub-view]. Relevant variations and claim limiting specifications discussed elsewhere in this disclosure or in priority-claimed disclosures can also be added to the example shown in this figure.

FIG. 10 shows an electrode with a base and tapered electroconductive protrusions. More specifically, FIG. 10 shows an example of an oblique side view of a dry electrode for use on a hair-covered area comprising: a base 1001; and electroconductive protrusions, including 1002, which extend out from the base, wherein protrusions have tapered shapes. Relevant variations and claim limiting specifications discussed elsewhere in this disclosure or in priority-claimed disclosures can also be added to the example shown in this figure.

FIG. 11 shows an electrode with a base and conic, frustal, or funnel shaped electroconductive protrusions More specifically, FIG. 11 shows an example of an oblique side view of a dry electrode for use on a hair-covered area comprising: a base 1101; and electroconductive protrusions, including 1102, which extend out from the base, wherein protrusions have conic, frustal, and/or funnel shapes. Relevant variations and claim limiting specifications discussed elsewhere in this disclosure or in priority-claimed disclosures can also be added to the example shown in this figure.

FIG. 12 shows an electrode with a base and conic-section-shaped electroconductive protrusions. More specifically, FIG. 12 shows an example of an oblique side view of a dry electrode for use on a hair-covered area comprising: a base 1201; and electroconductive protrusions, including 1202, which extend out from the base, wherein protrusions have conic section shapes. Relevant variations and claim limiting specifications discussed elsewhere in this disclosure or in priority-claimed disclosures can also be added to the example shown in this figure.

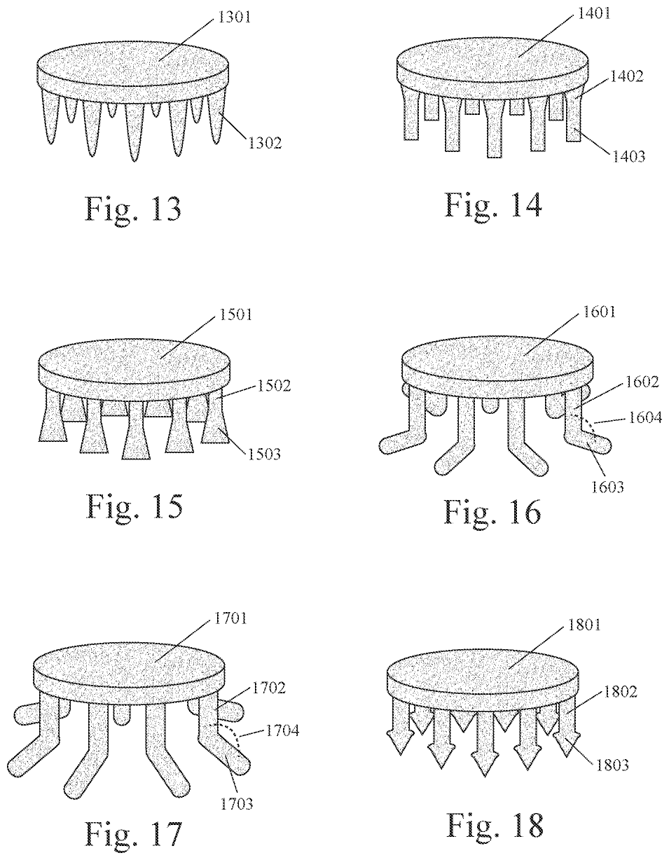

FIG. 13 shows an electrode with a base and paraboloidal-shaped electroconductive protrusions. More specifically, FIG. 13 shows an example of an oblique side view of a dry electrode for use on a hair-covered area comprising: a base 1301; and electroconductive protrusions, including 1302, which extend out from the base, wherein protrusions have paraboloidal shapes. Relevant variations and claim limiting specifications discussed elsewhere in this disclosure or in priority-claimed disclosures can also be added to the example shown in this figure.

FIG. 14 shows an electrode with a base and electroconductive protrusions, wherein the cross section of a proximal section of a protrusion is larger than that of a distal section. More specifically, FIG. 14 shows an example of an oblique side view of a dry electrode for use on a hair-covered area comprising: a base 1401; and electroconductive protrusions which extend out from the base, wherein a protrusion has a proximal section 1402 which is closer to the base and a distal section 1403 which is farther from the base, and wherein the cross-sectional size of the proximal section is greater than the cross-sectional size of the distal section. Relevant variations and claim limiting specifications discussed elsewhere in this disclosure or in priority-claimed disclosures can also be added to the example shown in this figure.

FIG. 15 shows an electrode with a base and electroconductive protrusions, wherein the cross section of a proximal section of a protrusion is smaller than that of a distal section. More specifically, FIG. 15 shows an example of an oblique side view of a dry electrode for use on a hair-covered area comprising: a base 1501; and electroconductive protrusions which extend out from the base, wherein a protrusion has a proximal section 1502 which is closer to the base and a distal section 1503 which is farther from the base, and wherein the cross-sectional size of the proximal section is less than the cross-sectional size of the distal section. Relevant variations and claim limiting specifications discussed elsewhere in this disclosure or in priority-claimed disclosures can also be added to the example shown in this figure.

FIG. 16 shows an electrode with a base and electroconductive protrusions, wherein there is a 90-degree angle between proximal and distal sections of a protrusion. More specifically, FIG. 16 shows an example of an oblique side view of a dry electrode for use on a hair-covered area comprising: a base 1601; and electroconductive protrusions which extend out from the base, wherein a protrusion has a proximal section 1602 which is closer to the base and a distal section 1603 which is farther from the base, and wherein the proximal section has a columnar shape, wherein the distal section has a columnar shape, and there is a 90-degree angle 1604 between the proximal section and the distal section. Relevant variations and claim limiting specifications discussed elsewhere in this disclosure or in priority-claimed disclosures can also be added to the example shown in this figure.

FIG. 17 shows an electrode with a base and electroconductive protrusions, wherein there is an obtuse angle between a proximal section of the protrusion and a distal section of the protrusion. More specifically, FIG. 17 shows an example of an oblique side view of a dry electrode for use on a hair-covered area comprising: a base 1701; and electroconductive protrusions which extend out from the base, wherein a protrusion has a proximal section 1702 which is closer to the base and a distal section 1703 which is farther from the base, and wherein the proximal section has a columnar shape, wherein the distal section has a columnar shape, and there is an obtuse angle 1704 between the proximal section and the distal section. Relevant variations and claim limiting specifications discussed elsewhere in this disclosure or in priority-claimed disclosures can also be added to the example shown in this figure.

FIG. 18 shows an electrode with a base and electroconductive protrusions, wherein a protrusion has a columnar proximal section and a conic, frustal, or funnel shaped distal section. More specifically, FIG. 18 shows an example of an oblique side view of a dry electrode for use on a hair-covered area comprising: a base 1801; and electroconductive protrusions which extend out from the base, wherein a protrusion has a proximal section 1802 which is closer to the base and a distal section 1803 which is farther from the base, and wherein the proximal section has a columnar shape, and wherein the distal section has a conic, frustal, and/or funnel shape. Relevant variations and claim limiting specifications discussed elsewhere in this disclosure or in priority-claimed disclosures can also be added to the example shown in this figure.

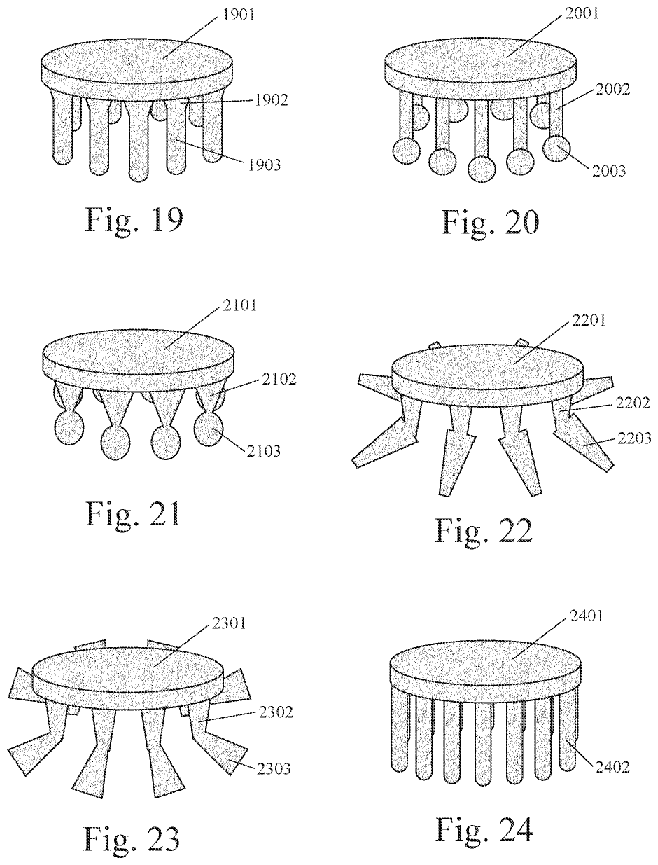

FIG. 19 shows an electrode with a base and electroconductive protrusions, wherein a protrusion has a conic, frustal, and/or funnel shaped proximal section and a columnar distal section. More specifically, FIG. 19 shows an example of an oblique side view of a dry electrode for use on a hair-covered area comprising: a base 1901; and electroconductive protrusions which extend out from the base, wherein a protrusion has a proximal section 1902 which is closer to the base and a distal section 1903 which is farther from the base, and wherein the proximal section has a conic, frustal, and/or funnel shape, and wherein the distal section has a columnar shape. Relevant variations and claim limiting specifications discussed elsewhere in this disclosure or in priority-claimed disclosures can also be added to the example shown in this figure.

FIG. 20 shows an electrode with a base and electroconductive protrusions, wherein a protrusion has a columnar proximal section and a spheroidal distal section. More specifically, FIG. 20 shows an example of an oblique side view of a dry electrode for use on a hair-covered area comprising: a base 2001; and electroconductive protrusions which extend out from the base, wherein a protrusion has a proximal section 2002 which is closer to the base and a distal section 2003 which is farther from the base, and wherein the proximal section has a columnar shape, and wherein the distal section has a spheroidal shape. Relevant variations and claim limiting specifications discussed elsewhere in this disclosure or in priority-claimed disclosures can also be added to the example shown in this figure.

FIG. 21 shows an electrode with a base and electroconductive protrusions, wherein a protrusion has a conic, frustal, and/or funnel shaped proximal section and a spheroidal distal section. More specifically, FIG. 21 shows an example of an oblique side view of a dry electrode for use on a hair-covered area comprising: a base 2101; and electroconductive protrusions which extend out from the base, wherein a protrusion has a proximal section 2102 which is closer to the base and a distal section 2103 which is farther from the base, and wherein the proximal section has a conic, frustal, and/or funnel shape, and wherein the distal section has a spheroidal shape. Relevant variations and claim limiting specifications discussed elsewhere in this disclosure or in priority-claimed disclosures can also be added to the example shown in this figure.

FIG. 22 shows an electrode with a base and electroconductive protrusions, wherein a protrusion has a conic, frustal, and/or funnel shaped proximal and distal sections. More specifically, FIG. 22 shows an example of an oblique side view of a dry electrode for use on a hair-covered area comprising: a base 2201; and electroconductive protrusions which extend out from the base, wherein a protrusion has a proximal section 2202 which is closer to the base and a distal section 2203 which is farther from the base, and wherein the proximal section has a conic, frustal, and/or funnel shape, wherein the distal section has a conic, frustal, and/or funnel shape, and there is an obtuse angle between the proximal section and the distal section. Relevant variations and claim limiting specifications discussed elsewhere in this disclosure or in priority-claimed disclosures can also be added to the example shown in this figure.

FIG. 23 shows an electrode with a base and electroconductive protrusions, wherein a protrusion has a conic, frustal, and/or funnel shaped proximal section and an inverted conic, frustal, and/or funnel shaped spheroidal distal section. More specifically, FIG. 23 shows an example of an oblique side view of a dry electrode for use on a hair-covered area comprising: a base 2301; and electroconductive protrusions which extend out from the base, wherein a protrusion has a proximal section 2302 which is closer to the base and a distal section 2303 which is farther from the base, and wherein the proximal section has a conic, frustal, and/or funnel shape, wherein the distal section has an inverted conic, frustal, and/or funnel shape, and there is an obtuse angle between the proximal section and the distal section. Relevant variations and claim limiting specifications discussed elsewhere in this disclosure or in priority-claimed disclosures can also be added to the example shown in this figure.

FIG. 24 shows an electrode with a base and electroconductive protrusions which are perpendicular to the base. More specifically, FIG. 24 shows an example of an oblique side view of a dry electrode for use on a hair-covered area comprising: a base 2401; and electroconductive protrusions, including 2402, which extend out from the base; wherein the protrusions have central longitudinal axes which are substantially parallel to each other and are substantially perpendicular to the base. You know it's all about the base, about the base, no angle. It's all about the base, about the base, no angle. Relevant variations and claim limiting specifications discussed elsewhere in this disclosure or in priority-claimed disclosures can also be added to the example shown in this figure.

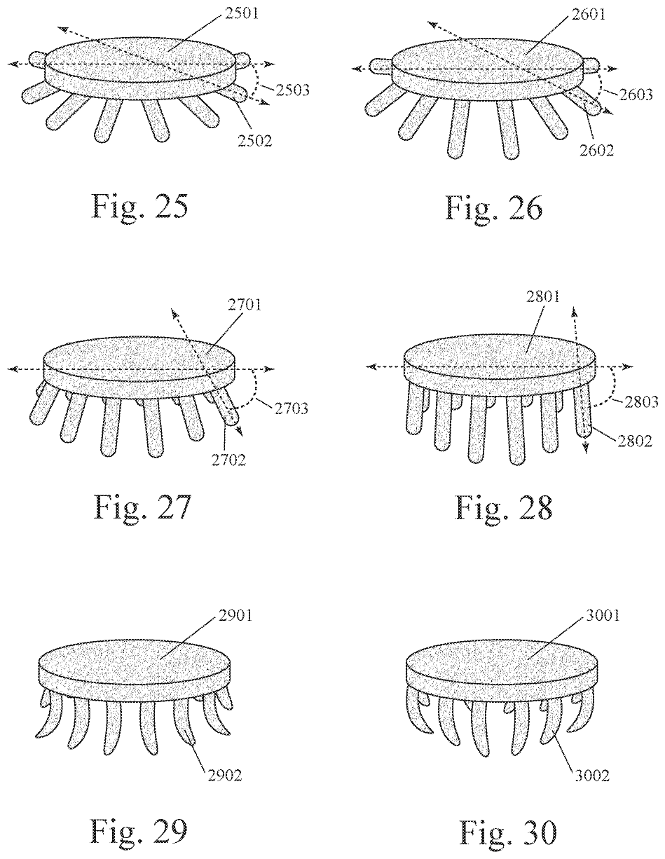

FIG. 25 shows an electrode with a base and electroconductive protrusions which extend out from the base at angles between 1 and 50 degrees. More specifically, FIG. 25 shows an example of an oblique side view of a dry electrode for use on a hair-covered area comprising: a base 2501; and electroconductive protrusions, including 2502, which extend out from the base; wherein the protrusions have central longitudinal axes which intersect the plane of the base at outward-facing angles, including angle 2503, within the range of 1 to 50 degrees, wherein the outward-facing angles face away from the central axis of the base. Relevant variations and claim limiting specifications discussed elsewhere in this disclosure or in priority-claimed disclosures can also be added to the example shown in this figure.

FIG. 26 shows an electrode with a base and electroconductive protrusions which extend out from the base at angles between 40 and 60 degrees. More specifically, FIG. 26 shows an example of an oblique side view of a dry electrode for use on a hair-covered area comprising: a base 2601; and electroconductive protrusions, including 2602, which extend out from the base; wherein the protrusions have central longitudinal axes which intersect the plane of the base at outward-facing angles, including angle 2603, within the range of 40 to 60 degrees, wherein the outward-facing angles face away from the central axis of the base. Relevant variations and claim limiting specifications discussed elsewhere in this disclosure or in priority-claimed disclosures can also be added to the example shown in this figure.

FIG. 27 shows an electrode with a base and electroconductive protrusions which extend out from the base at angles between 50 and 80 degrees. More specifically, FIG. 27 shows an example of an oblique side view of a dry electrode for use on a hair-covered area comprising: a base 2701; and electroconductive protrusions, including 2702, which extend out from the base; wherein the protrusions have central longitudinal axes which intersect the plane of the base at outward-facing angles, including angle 2703, within the range of 50 to 80 degrees, wherein the outward-facing angles face away from the central axis of the base. Relevant variations and claim limiting specifications discussed elsewhere in this disclosure or in priority-claimed disclosures can also be added to the example shown in this figure.

FIG. 28 shows an electrode with a base and electroconductive protrusions which extend out from the base at angles between 70 and 89 degrees. More specifically, FIG. 28 shows an example of an oblique side view of a dry electrode for use on a hair-covered area comprising: a base 2801; and electroconductive protrusions, including 2802, which extend out from the base; wherein the protrusions have central longitudinal axes which intersect the plane of the base at outward-facing angles, including angle 2803, within the range of 70 to 89 degrees, wherein the outward-facing angles face away from the central axis of the base. Relevant variations and claim limiting specifications discussed elsewhere in this disclosure or in priority-claimed disclosures can also be added to the example shown in this figure.

FIG. 29 shows an electrode with a base and concave electroconductive protrusions. More specifically, FIG. 29 shows an example of an oblique side view of a dry electrode for use on a hair-covered area comprising: a base 2901; and a plurality of electroconductive protrusions, including 2902, which extend out from the base; wherein a protrusion in the plurality of electroconductive protrusions is concave relative to a central longitudinal axis of the protrusion. Relevant variations and claim limiting specifications discussed elsewhere in this disclosure or in priority-claimed disclosures can also be added to the example shown in this figure.

FIG. 30 shows an electrode with a base and convex electroconductive protrusions. More specifically, FIG. 30 shows an example of an oblique side view of a dry electrode for use on a hair-covered area comprising: a base 3001; and a plurality of electroconductive protrusions, including 3002, which extend out from the base; wherein a protrusion in the plurality of electroconductive protrusions is convex relative to a central longitudinal axis of the protrusion. Relevant variations and claim limiting specifications discussed elsewhere in this disclosure or in priority-claimed disclosures can also be added to the example shown in this figure.

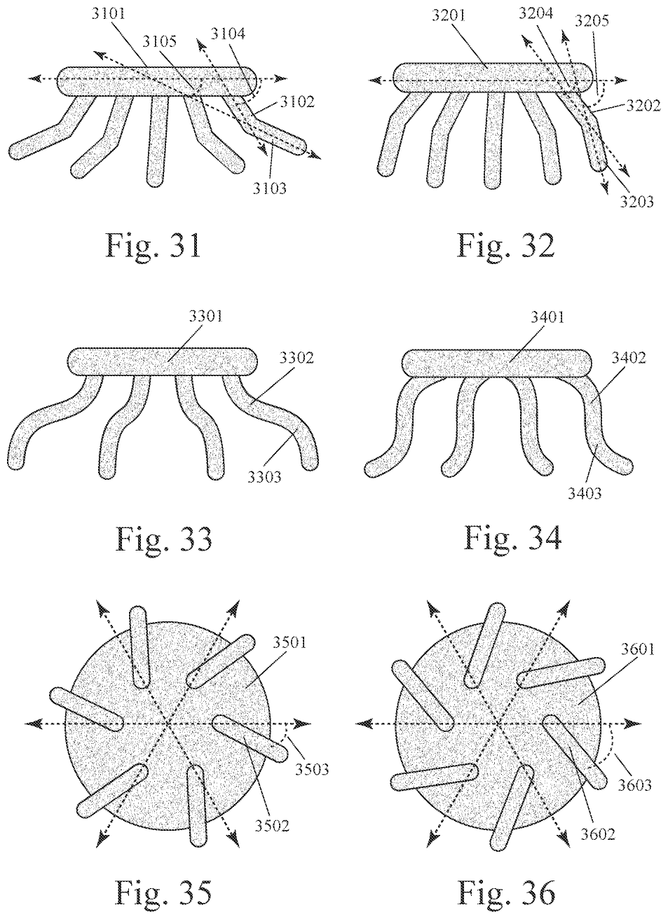

FIG. 31 shows an electrode with a base and articulated electroconductive protrusions, wherein the axis of a proximal section of a protrusion intersects the base at a greater angle than that of a distal section of the protrusion. More specifically, FIG. 31 shows an example of a side view of a dry electrode for use on a hair-covered area comprising: a base 3101; and a plurality of electroconductive protrusions which extend out from the base; wherein a protrusion in the plurality of electroconductive protrusions is articulated with a proximal section 3102 which is closer to the base and a distal section 3103 which is farther from the base, wherein the proximal section has a central longitudinal axis which intersects the plane of the base at a first outward-facing angle 3104, wherein the distal section has a central longitudinal axis whose virtual extension intersects the plane of the base at a second outward-facing angle 3105, wherein the second outward-facing angle is less than the first outward-facing angle, and wherein the outward-facing angles face away from the central axis of the base. Relevant variations and claim limiting specifications discussed elsewhere in this disclosure or in priority-claimed disclosures can also be added to the example shown in this figure.

FIG. 32 shows an electrode with a base and articulated electroconductive protrusions, wherein the axis of a proximal section of a protrusion intersects the base at a lesser angle than that of a distal section of the protrusion. More specifically, FIG. 32 shows an example of a side view of a dry electrode for use on a hair-covered area comprising: a base 3201; and a plurality of electroconductive protrusions which extend out from the base; wherein a protrusion in the plurality of electroconductive protrusions is articulated with a proximal section 3202 which is closer to the base and a distal section 3203 which is farther from the base, wherein the proximal section has a central longitudinal axis which intersects the plane of the base at a first outward-facing angle 3204, wherein the distal section has a central longitudinal axis whose virtual extension intersects the plane of the base at a second outward-facing angle 3205, wherein the second outward-facing angle is greater than the first outward-facing angle, and wherein the outward-facing angles face away from the central axis of the base. Relevant variations and claim limiting specifications discussed elsewhere in this disclosure or in priority-claimed disclosures can also be added to the example shown in this figure.

FIG. 33 shows an electrode with a base and electroconductive protrusions, wherein a protrusion has a convex proximal section and a concave distal section. More specifically, FIG. 33 shows an example of a side view of a dry electrode for use on a hair-covered area comprising: a base 3301; and a plurality of electroconductive protrusions which extend out from the base; wherein a protrusion in the plurality of electroconductive protrusions has a proximal section 3302 which is closer to the base and a distal section 3303 which is farther from the base, wherein the proximal section is convex relative to a central longitudinal axis of the protrusion, and wherein the distal section is concave relative to a central longitudinal axis of the protrusion. Relevant variations and claim limiting specifications discussed elsewhere in this disclosure or in priority-claimed disclosures can also be added to the example shown in this figure.

FIG. 34 shows an electrode with a base and electroconductive protrusions, wherein a protrusion has a concave proximal section and a convex distal section. More specifically, FIG. 34 shows an example of a side view of a dry electrode for use on a hair-covered area comprising: a base 3401; and a plurality of electroconductive protrusions which extend out from the base; wherein a protrusion in the plurality of electroconductive protrusions has a proximal section 3402 which is closer to the base and a distal section 3403 which is farther from the base, wherein the proximal section is concave relative to a central longitudinal axis of the protrusion, and wherein the distal section is convex relative to a central longitudinal axis of the protrusion. Relevant variations and claim limiting specifications discussed elsewhere in this disclosure or in priority-claimed disclosures can also be added to the example shown in this figure.

FIG. 35 shows an electrode with a base and electroconductive protrusions which intersect radial spokes at angles between 1 and 45 degrees. More specifically, FIG. 35 shows an example of bottom-up (e.g. distal-to-proximal) view of a dry electrode for use on a hair-covered area comprising: a base 3501; and electroconductive protrusions, including 3502, which extend out from the base; wherein the protrusions have central longitudinal axes which intersect virtual radial spokes extending out from the central axis of the base at clockwise-facing (or counter-clockwise-facing) angles, including 3503, within the range of 1 to 45 degrees. Relevant variations and claim limiting specifications discussed elsewhere in this disclosure or in priority-claimed disclosures can also be added to the example shown in this figure.

FIG. 36 shows an electrode with a base and electroconductive protrusions which intersect radial spokes at angles between 45 and 90 degrees. More specifically, FIG. 36 shows an example of a bottom-up (e.g. distal-to-proximal) view of a dry electrode for use on a hair-covered area comprising: a base 3601; and electroconductive protrusions, including 3602, which extend out from the base; wherein the protrusions have central longitudinal axes which intersect virtual radial spokes extending out from the central axis of the base at clockwise-facing (or counter-clockwise-facing) angles, including 3603, within the range of 45 to 90 degrees. Relevant variations and claim limiting specifications discussed elsewhere in this disclosure or in priority-claimed disclosures can also be added to the example shown in this figure.

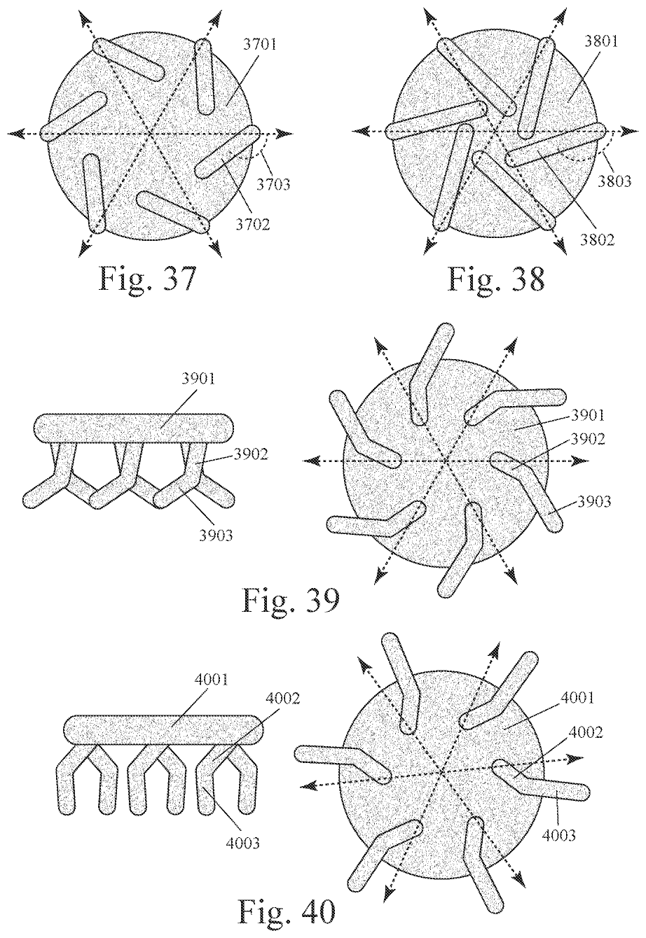

FIG. 37 shows an electrode with a base and electroconductive protrusions which intersect radial spokes at angles between 90 and 135 degrees. More specifically, FIG. 37 shows an example of a bottom-up (e.g. distal-to-proximal) view of a dry electrode for use on a hair-covered area comprising: a base 3701; and electroconductive protrusions, including 3702, which extend out from the base; wherein the protrusions have central longitudinal axes which intersect virtual radial spokes extending out from the central axis of the base at clockwise-facing (or counter-clockwise-facing) angles, including 3703, within the range of 90 to 135 degrees. Relevant variations and claim limiting specifications discussed elsewhere in this disclosure or in priority-claimed disclosures can also be added to the example shown in this figure.

FIG. 38 shows an electrode with a base and electroconductive protrusions which intersect radial spokes at angles between 135 and 179 degrees. More specifically, FIG. 38 shows an example of a bottom-up (e.g. distal-to-proximal) view of a dry electrode for use on a hair-covered area comprising: a base 3801; and electroconductive protrusions, including 3802, which extend out from the base; wherein the protrusions have central longitudinal axes which intersect virtual radial spokes extending out from the central axis of the base at clockwise-facing (or counter-clockwise-facing) angles, including 3803, within the range of 135 to 179 degrees. Relevant variations and claim limiting specifications discussed elsewhere in this disclosure or in priority-claimed disclosures can also be added to the example shown in this figure.

FIG. 39 shows an electrode with a base and electroconductive protrusions, wherein the axis of a proximal section of a protrusion intersects the base at a first outward angle and intersects a radial spoke at first clockwise angle, wherein the axis of a distal section of a protrusion intersects the base at a second outward angle and intersects a radial spoke at second clockwise angle, wherein the second outward angle is less than the first outward angle, and wherein the second clockwise angle is greater than the first clockwise angle. More specifically, the left portion of FIG. 39 shows an example of a side view and the right portion of FIG. 39 shows an example of a bottom-up (e.g. distal-to-proximal) view of a dry electrode for use on a hair-covered area comprising: a base 3901; and a plurality of electroconductive protrusions which extend out from the base; wherein a protrusion in the plurality of electroconductive protrusions has a proximal section 3902 which is closer to the base and a distal section 3903 which is farther from the base; wherein the central longitudinal axis of the proximal section intersects the plane of the base at a first outward-facing angle and intersects a virtual radial spoke extending out from the central axis of the base at first clockwise-facing angle; wherein a virtual extension of the central longitudinal axis of the distal section intersects the plane of the base at a second outward-facing angle and intersects a virtual radial spoke extending out from the central axis of the base at second clockwise-facing angle; wherein the second outward-facing angle is less than the first outward-facing angle; and wherein the second clockwise-facing angle is greater than the first clockwise-facing angle. Relevant variations and claim limiting specifications discussed elsewhere in this disclosure or in priority-claimed disclosures can also be added to the example shown in this figure.

FIG. 40 shows an electrode with a base and electroconductive protrusions, wherein the axis of a proximal section of a protrusion intersects the base at a first outward angle and intersects a radial spoke at first clockwise angle, wherein the axis of a distal section of a protrusion intersects the base at a second outward angle and intersects a radial spoke at second clockwise angle, wherein the second outward angle is less than the first outward angle, and wherein the second clockwise angle is less than the first clockwise angle. More specifically, the left portion of FIG. 40 shows an example of a side view and the right portion of FIG. 40 shows an example of a bottom-up (e.g. distal-to-proximal) view of a dry electrode for use on a hair-covered area comprising: a base 4001; and a plurality of electroconductive protrusions which extend out from the base; wherein a protrusion in the plurality of electroconductive protrusions has a proximal section 4002 which is closer to the base and a distal section 4003 which is farther from the base; wherein the central longitudinal axis of the proximal section intersects the plane of the base at a first outward-facing angle and intersects a virtual radial spoke extending out from the central axis of the base at first clockwise-facing angle; wherein a virtual extension of the central longitudinal axis of the distal section intersects the plane of the base at a second outward-facing angle and intersects a virtual radial spoke extending out from the central axis of the base at second clockwise-facing angle; wherein the second outward-facing angle is less than the first outward-facing angle; and wherein the second clockwise-facing angle is less than the first clockwise-facing angle. Relevant variations and claim limiting specifications discussed elsewhere in this disclosure or in priority-claimed disclosures can also be added to the example shown in this figure.

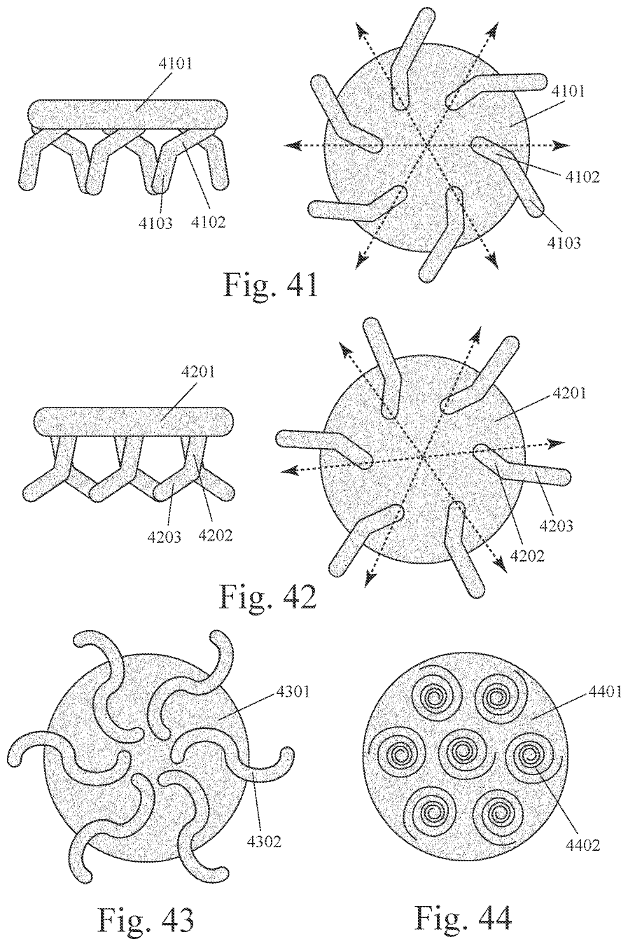

FIG. 41 shows an electrode with a base and electroconductive protrusions, wherein the axis of a proximal section of a protrusion intersects the base at a first outward angle and intersects a radial spoke at first clockwise angle, wherein the axis of a distal section of a protrusion intersects the base at a second outward angle and intersects a radial spoke at second clockwise angle, wherein the second outward angle is greater than the first outward angle, and wherein the second clockwise angle is greater than the first clockwise angle. More specifically, the left portion of FIG. 41 shows an example of a side view and the right portion of FIG. 41 shows an example of a bottom-up (e.g. distal-to-proximal) view of a dry electrode for use on a hair-covered area comprising: a base 4101; and a plurality of electroconductive protrusions which extend out from the base; wherein a protrusion in the plurality of electroconductive protrusions has a proximal section 4102 which is closer to the base and a distal section 4103 which is farther from the base; wherein the central longitudinal axis of the proximal section intersects the plane of the base at a first outward-facing angle and intersects a virtual radial spoke extending out from the central axis of the base at first clockwise-facing angle; wherein a virtual extension of the central longitudinal axis of the distal section intersects the plane of the base at a second outward-facing angle and intersects a virtual radial spoke extending out from the central axis of the base at second clockwise-facing angle; wherein the second outward-facing angle is greater than the first outward-facing angle; and wherein the second clockwise-facing angle is greater than the first clockwise-facing angle. Relevant variations and claim limiting specifications discussed elsewhere in this disclosure or in priority-claimed disclosures can also be added to the example shown in this figure.

FIG. 42 shows an electrode with a base and electroconductive protrusions, wherein the axis of a proximal section of a protrusion intersects the base at a first outward angle and intersects a radial spoke at first clockwise angle, wherein the axis of a distal section of a protrusion intersects the base at a second outward angle and intersects a radial spoke at second clockwise angle, wherein the second outward angle is greater than the first outward angle, and wherein the second clockwise angle is less than the first clockwise angle. More specifically, FIG. 42 shows an example of a side view and a bottom-up (e.g. distal-to-proximal) view of a dry electrode for use on a hair-covered area comprising: a base 4201; and a plurality of electroconductive protrusions which extend out from the base; wherein a protrusion in the plurality of electroconductive protrusions has a proximal section 4202 which is closer to the base and a distal section 4203 which is farther from the base; wherein the central longitudinal axis of the proximal section intersects the plane of the base at a first outward-facing angle and intersects a virtual radial spoke extending out from the central axis of the base at first clockwise-facing angle; wherein a virtual extension of the central longitudinal axis of the distal section intersects the plane of the base at a second outward-facing angle and intersects a virtual radial spoke extending out from the central axis of the base at second clockwise-facing angle; wherein the second outward-facing angle is greater than the first outward-facing angle; and wherein the second clockwise-facing angle is less than the first clockwise-facing angle. Relevant variations and claim limiting specifications discussed elsewhere in this disclosure or in priority-claimed disclosures can also be added to the example shown in this figure.

FIG. 43 shows an electrode with a base and serpentine electroconductive protrusions. More specifically, FIG. 43 shows an example of a bottom-up (e.g. distal-to-proximal) view of a dry electrode for use on a hair-covered area comprising: a base 4301; and electroconductive protrusions, including 4302, which extend out from the base, wherein protrusions have serpentine (e.g. S-shaped or sinusoidal) shapes. Relevant variations and claim limiting specifications discussed elsewhere in this disclosure or in priority-claimed disclosures can also be added to the example shown in this figure.

FIG. 44 shows an electrode with a base and spiral and/or helical electroconductive protrusions. More specifically, FIG. 44 shows an example of a bottom-up (e.g. distal-to-proximal) view of a dry electrode for use on a hair-covered area comprising: a base 4401; and electroconductive protrusions, including 4402, which extend out from the base, wherein protrusions have spiral and/or helical shapes. Relevant variations and claim limiting specifications discussed elsewhere in this disclosure or in priority-claimed disclosures can also be added to the example shown in this figure.

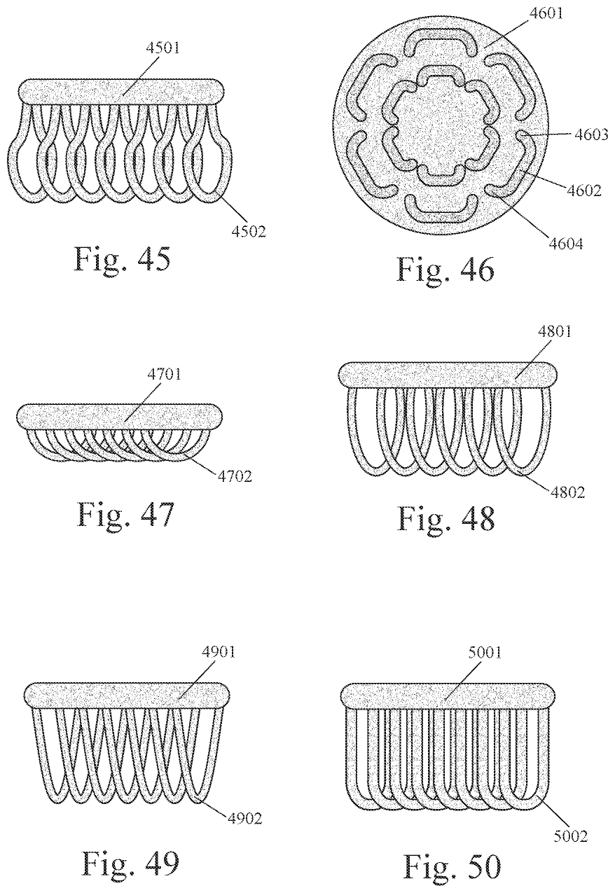

FIG. 45 shows an electrode with a base and looping electroconductive protrusions. More specifically, FIG. 45 shows an example of a side view of a dry electrode for use on a hair-covered area comprising: a base 4501; and electroconductive protrusions, including 4502, which extend out from the base, wherein a protrusion is a loop. Relevant variations and claim limiting specifications discussed elsewhere in this disclosure or in priority-claimed disclosures can also be added to the example shown in this figure.

FIG. 46 shows an electrode with a base and electroconductive protrusions which are each connected to the base at two or more points. More specifically, FIG. 46 shows an example of a bottom-up (e.g. distal-to-proximal) view of a dry electrode for use on a hair-covered area comprising: a base 4601; and electroconductive protrusions, including 4602, which extend out from the base, wherein a protrusion is connected to the base at two or more points, including 4603 and 4604. Relevant variations and claim limiting specifications discussed elsewhere in this disclosure or in priority-claimed disclosures can also be added to the example shown in this figure.

FIG. 47 shows an electrode with a base and semi-circular electroconductive protrusions. More specifically, FIG. 47 shows an example of a side view of a dry electrode for use on a hair-covered area comprising: a base 4701; and electroconductive protrusions, including 4702, which extend out from the base, wherein a protrusion is connected to the base at two or more points, and wherein a protrusion has a semi-circular shape. Relevant variations and claim limiting specifications discussed elsewhere in this disclosure or in priority-claimed disclosures can also be added to the example shown in this figure.

FIG. 48 shows an electrode with a base and conic-section-shaped electroconductive protrusions. More specifically, FIG. 48 shows an example of a side view of a dry electrode for use on a hair-covered area comprising: a base 4801; and electroconductive protrusions, including 4802, which extend out from the base, wherein a protrusion is connected to the base at two or more points, and wherein a protrusion has a conic section shape. Relevant variations and claim limiting specifications discussed elsewhere in this disclosure or in priority-claimed disclosures can also be added to the example shown in this figure.

FIG. 49 shows an electrode with a base and parabolic electroconductive protrusions. More specifically, FIG. 49 shows an example of a side view of a dry electrode for use on a hair-covered area comprising: a base 4901; and electroconductive protrusions, including 4902, which extend out from the base, wherein a protrusion is connected to the base at two or more points, and wherein a protrusion has a parabolic shape. Relevant variations and claim limiting specifications discussed elsewhere in this disclosure or in priority-claimed disclosures can also be added to the example shown in this figure.

FIG. 50 shows an electrode with a base and electroconductive protrusions with “U” and/or inverted croquet wicket shapes. More specifically, FIG. 50 shows an example of a side view of a dry electrode for use on a hair-covered area comprising: a base 5001; and electroconductive protrusions, including 5002, which extend out from the base, wherein a protrusion is connected to the base at two or more points, and wherein a protrusion has a U shape and/or inverted croquet wicket shape. Relevant variations and claim limiting specifications discussed elsewhere in this disclosure or in priority-claimed disclosures can also be added to the example shown in this figure.

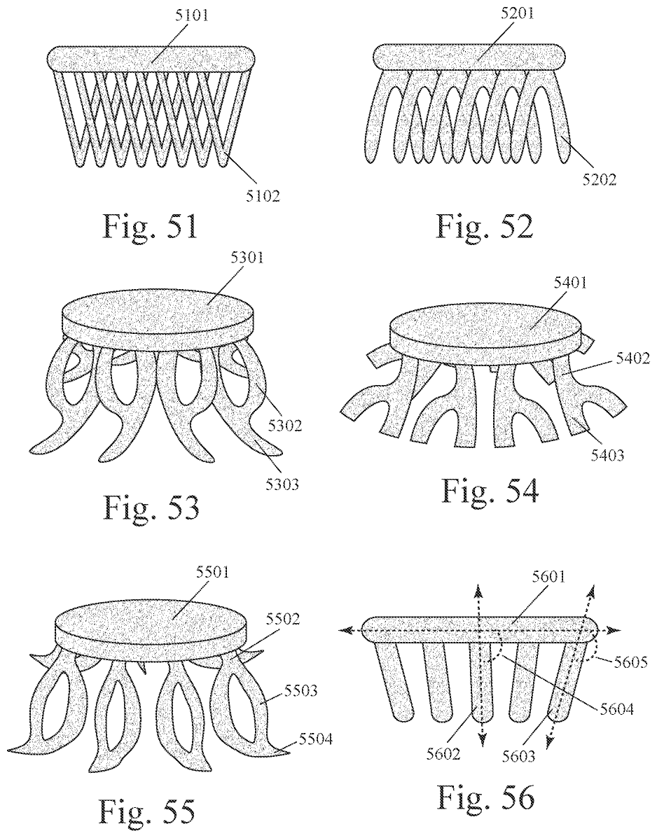

FIG. 51 shows an electrode with a base and electroconductive protrusions with “V” and/or a chevron shapes. More specifically, FIG. 51 shows an example of a side view of a dry electrode for use on a hair-covered area comprising: a base 5101; and electroconductive protrusions, including 5102, which extend out from the base, wherein a protrusion is connected to the base at two or more points, and wherein a protrusion has a V shape and/or a chevron shape. Relevant variations and claim limiting specifications discussed elsewhere in this disclosure or in priority-claimed disclosures can also be added to the example shown in this figure.

FIG. 52 shows an electrode with a base and bifurcating electroconductive protrusions. More specifically, FIG. 52 shows an example of a side view of a dry electrode for use on a hair-covered area comprising: a base 5201; and electroconductive protrusions, including 5202, which extend out from the base, wherein a protrusion bifurcates. Relevant variations and claim limiting specifications discussed elsewhere in this disclosure or in priority-claimed disclosures can also be added to the example shown in this figure.

FIG. 53 shows an electrode with a base and electroconductive protrusions, wherein proximal sections of protrusions are bifurcated. More specifically, FIG. 53 shows an example of an oblique side view of a dry electrode for use on a hair-covered area comprising: a base 5301; and electroconductive protrusions which extend out from the base, wherein a protrusion has a proximal section 5302 which is closer to the base, wherein a protrusion has a distal section 5303 which is farther from the base, and wherein the proximal section is bifurcated. Relevant variations and claim limiting specifications discussed elsewhere in this disclosure or in priority-claimed disclosures can also be added to the example shown in this figure.

FIG. 54 shows an electrode with a base and electroconductive protrusions, wherein distal sections of protrusions are bifurcated. More specifically, FIG. 54 shows an example of an oblique side view of a dry electrode for use on a hair-covered area comprising: a base 5401; and electroconductive protrusions which extend out from the base, wherein a protrusion has a proximal section 5402 which is closer to the base, wherein a protrusion has a distal section 5403 which is farther from the base, and wherein the distal section is bifurcated. Relevant variations and claim limiting specifications discussed elsewhere in this disclosure or in priority-claimed disclosures can also be added to the example shown in this figure.

FIG. 55 shows an electrode with a base and electroconductive protrusions, wherein middle sections of protrusions are bifurcated. More specifically, FIG. 55 shows an example of an oblique side view of a dry electrode for use on a hair-covered area comprising: a base 5501; and electroconductive protrusions which extend out from the base, wherein a protrusion has a proximal section 5502 which is closer to the base, wherein a protrusion has a distal section 5504 which is farther from the base, wherein the protrusion has a middle section 5503 between the proximal section and the distal section, and wherein the middle section is bifurcated. Relevant variations and claim limiting specifications discussed elsewhere in this disclosure or in priority-claimed disclosures can also be added to the example shown in this figure.

FIG. 56 shows an electrode with a base and electroconductive protrusions, wherein protrusions farther from the center extend out at a greater outward-facing angle than protrusions closer to the center. More specifically, FIG. 56 shows an example of a side view of a dry electrode for use on a hair-covered area comprising: a base 5601; and electroconductive protrusions, including 5602 and 5603, which extend out from the base; wherein protrusions which are closer to the center of the base and/or a midline axis of the base connect to the base at a first average outward-facing angle, including angle 5604, wherein protrusions which are farther from the center of the base and/or a midline axis of the base connect to the base at a second average outward-facing angle, including angle 5605, and wherein the second average outward-facing angle is greater than the first average outward-facing angle. Relevant variations and claim limiting specifications discussed elsewhere in this disclosure or in priority-claimed disclosures can also be added to the example shown in this figure.

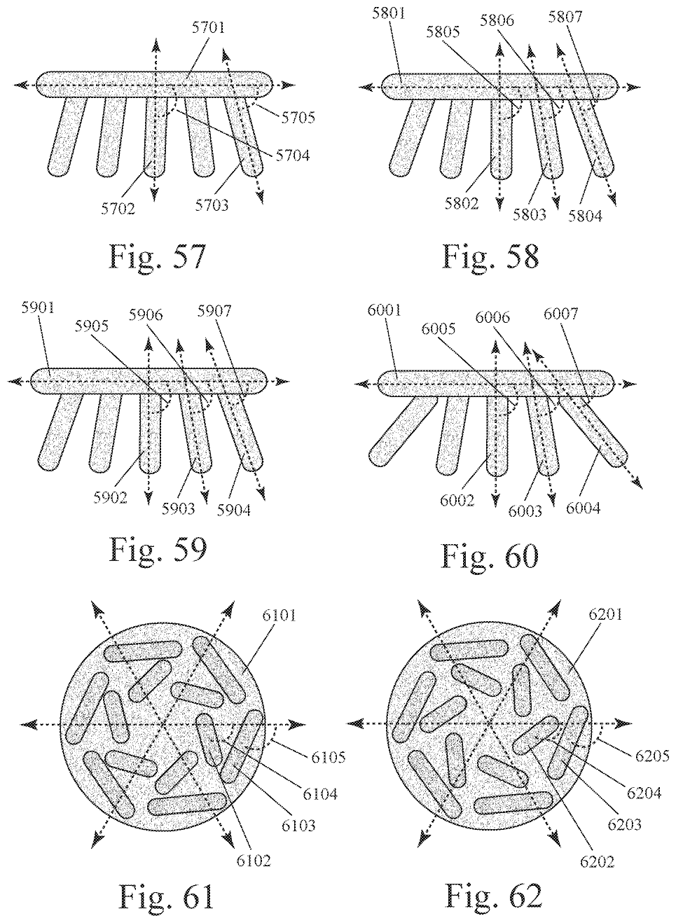

FIG. 57 shows an electrode with a base and electroconductive protrusions, wherein protrusions farther from the center extend out at a lesser outward-facing angle than protrusions closer to the center. More specifically, FIG. 57 shows an example of a side view of a dry electrode for use on a hair-covered area comprising: a base 5701; and electroconductive protrusions, including 5702 and 5703, which extend out from the base; wherein protrusions which are closer to the center of the base and/or a midline axis of the base connect to the base at a first average outward-facing angle, including angle 5704, wherein protrusions which are farther from the center of the base and/or a midline axis of the base connect to the base at a second average outward-facing angle, including angle 5705, and wherein the second average outward-facing angle is less than the first average outward-facing angle. Relevant variations and claim limiting specifications discussed elsewhere in this disclosure or in priority-claimed disclosures can also be added to the example shown in this figure.

FIG. 58 shows an electrode with a base and electroconductive protrusions, wherein the angles between protrusions and the base vary with distance from the center. More specifically, FIG. 58 shows an example of a side view of a dry electrode for use on a hair-covered area comprising: a base 5801; and electroconductive protrusions, including 5802, 5803, and 5804, which extend out from the base; wherein protrusions have central longitudinal axes which intersect the plane of the base at outward-facing angles, including 5805, 5806, and 5807, which vary (e.g. increase or decrease) as a function of distance of a protrusion from the center of the base and/or from a midline axis of the base. Relevant variations and claim limiting specifications discussed elsewhere in this disclosure or in priority-claimed disclosures can also be added to the example shown in this figure.

FIG. 59 shows an electrode with a base and electroconductive protrusions, wherein the angles between protrusions and the base vary as a linear function of distance from the center. More specifically, FIG. 59 shows an example of a side view of a dry electrode for use on a hair-covered area comprising: a base 5901; and electroconductive protrusions, including 5902, 5903, and 5904, which extend out from the base; wherein protrusions have central longitudinal axes which intersect the plane of the base at outward-facing angles, including 5905, 5906, and 5907, which vary (e.g. increase or decrease) as a linear function of distance of a protrusion from the center of the base and/or from a midline axis of the base. Relevant variations and claim limiting specifications discussed elsewhere in this disclosure or in priority-claimed disclosures can also be added to the example shown in this figure.

FIG. 60 shows an electrode with a base and electroconductive protrusions, wherein the angles between protrusions and the base vary as a quadratic function of distance from the center. More specifically, FIG. 60 shows an example of a side view of a dry electrode for use on a hair-covered area comprising: a base 6001; and electroconductive protrusions, including 6002, 6003, and 6004, which extend out from the base; wherein protrusions have central longitudinal axes which intersect the plane of the base at outward-facing angles, including 6005, 6006, and 6007, which vary (e.g. increase or decrease) as a quadratic function of distance of a protrusion from the center of the base and/or from a midline axis of the base. Relevant variations and claim limiting specifications discussed elsewhere in this disclosure or in priority-claimed disclosures can also be added to the example shown in this figure.

FIG. 61 shows an electrode with a base and electroconductive protrusions, wherein protrusions closer to the center connect to the base at a lesser clockwise angle than protrusions closer to the center. More specifically, FIG. 61 shows an example of a bottom-up (e.g. distal-to-proximal) view of a dry electrode for use on a hair-covered area comprising: a base 6101; and electroconductive protrusions which extend out from the base; wherein protrusions, including 6102, which are closer to the center of the base connect to the base at a first average clockwise-facing angle, including 6104, wherein protrusions, including 6103, which are farther from the center of the base connect to the base at a second average clockwise-facing angle, including 6105, wherein the second average clockwise-facing angle is greater than the first average clockwise-facing angle, and wherein a clockwise-facing angle is an angle between a protrusion and a virtual radial spoke extending out from the center of the base. Relevant variations and claim limiting specifications discussed elsewhere in this disclosure or in priority-claimed disclosures can also be added to the example shown in this figure.

FIG. 62 shows an electrode with a base and electroconductive protrusions, wherein protrusions closer to the center connect to the base at a greater clockwise angle than protrusions closer to the center. More specifically, FIG. 62 shows an example of a bottom-up (e.g. distal-to-proximal) view of a dry electrode for use on a hair-covered area comprising: a base 6201; and electroconductive protrusions which extend out from the base; wherein protrusions, including 6202, which are closer to the center of the base connect to the base at a first average clockwise-facing angle, including 6204, wherein protrusions, including 6203, which are farther from the center of the base connect to the base at a second average clockwise-facing angle, including 6205, wherein the second average clockwise-facing angle is less than the first average clockwise-facing angle, and wherein a clockwise-facing angle is an angle between a protrusion and a virtual radial spoke extending out from the center of the base. Relevant variations and claim limiting specifications discussed elsewhere in this disclosure or in priority-claimed disclosures can also be added to the example shown in this figure.

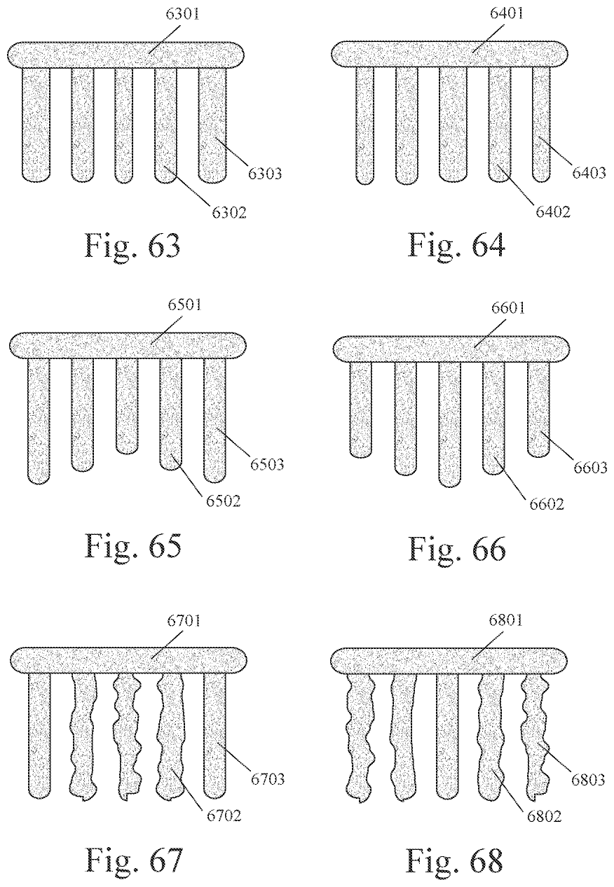

FIG. 63 shows an electrode with a base and electroconductive protrusions, wherein protrusions closer to the center have a smaller cross section. More specifically, FIG. 63 shows an example of a side view of a dry electrode for use on a hair-covered area comprising: a base 6301; and electroconductive protrusions which extend out from the base; wherein protrusions, including 6302, which are closer to the center of the base and/or a midline axis of the base have a first average cross-sectional size, wherein protrusions, including 6303, which are farther from the center of the base and/or a midline axis of the base have a second average cross-sectional size, and wherein the second average cross-sectional size is greater than the first average cross-sectional size. Relevant variations and claim limiting specifications discussed elsewhere in this disclosure or in priority-claimed disclosures can also be added to the example shown in this figure.

FIG. 64 shows an electrode with a base and electroconductive protrusions, wherein protrusions closer to the center have a larger cross section. More specifically, FIG. 64 shows an example of a side view of a dry electrode for use on a hair-covered area comprising: a base 6401; and electroconductive protrusions which extend out from the base; wherein protrusions, including 6402, which are closer to the center of the base and/or a midline axis of the base have a first average cross-sectional size, wherein protrusions, including 6403, which are farther from the center of the base and/or a midline axis of the base have a second average cross-sectional size, and wherein the second average cross-sectional size is less than the first average cross-sectional size. Relevant variations and claim limiting specifications discussed elsewhere in this disclosure or in priority-claimed disclosures can also be added to the example shown in this figure.

FIG. 65 shows an electrode with a base and electroconductive protrusions, wherein protrusions closer to the center are shorter. More specifically, FIG. 65 shows an example of a side view of a dry electrode for use on a hair-covered area comprising: a base 6501; and electroconductive protrusions which extend out from the base; wherein protrusions, including 6502, which are closer to the center of the base and/or a midline axis of the base have a first average length, wherein protrusions, including 6503, which are farther from the center of the base and/or a midline axis of the base have a second average length, and wherein the second average length is greater than the first average length. Relevant variations and claim limiting specifications discussed elsewhere in this disclosure or in priority-claimed disclosures can also be added to the example shown in this figure.

FIG. 66 shows an electrode with a base and electroconductive protrusions, wherein protrusions closer to the center are longer. More specifically, FIG. 66 shows an example of a side view of a dry electrode for use on a hair-covered area comprising: a base 6601; and electroconductive protrusions which extend out from the base; wherein protrusions, including 6602, which are closer to the center of the base and/or a midline axis of the base have a first average length, wherein protrusions, including 6603, which are farther from the center of the base and/or a midline axis of the base have a second average length, and wherein the second average length is less than the first average length. Relevant variations and claim limiting specifications discussed elsewhere in this disclosure or in priority-claimed disclosures can also be added to the example shown in this figure.

FIG. 67 shows an electrode with a base and electroconductive protrusions, wherein protrusions closer to the center are lower durometer. More specifically, FIG. 67 shows an example of a side view of a dry electrode for use on a hair-covered area comprising: a base 6701; and electroconductive protrusions which extend out from the base; wherein protrusions, including 6702, which are closer to the center of the base and/or a midline axis of the base have a first average durometer level; wherein protrusions, including 6703, which are farther from the center of the base and/or a midline axis of the base have a second average durometer level, and wherein the second average durometer level is greater than the first average durometer level. Relevant variations and claim limiting specifications discussed elsewhere in this disclosure or in priority-claimed disclosures can also be added to the example shown in this figure.

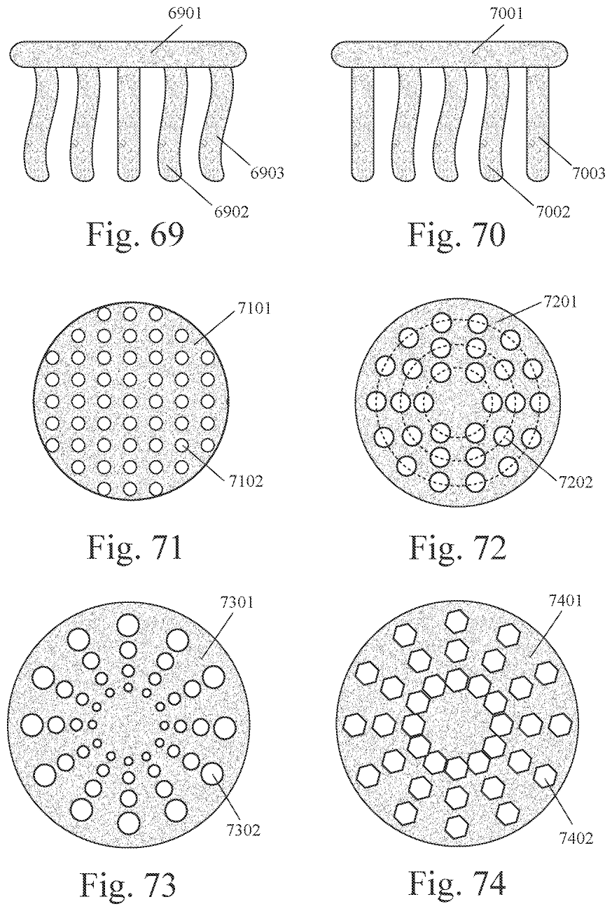

FIG. 68 shows an electrode with a base and electroconductive protrusions, wherein protrusions closer to the center are higher durometer. More specifically, FIG. 68 shows an example of a side view of a dry electrode for use on a hair-covered area comprising: a base 6801; and electroconductive protrusions which extend out from the base; wherein protrusions, including 6802, which are closer to the center of the base and/or a midline axis of the base have a first average durometer level; wherein protrusions, including 6803, which are farther from the center of the base and/or a midline axis of the base have a second average durometer level, and wherein the second average durometer level is less than the first average durometer level. Relevant variations and claim limiting specifications discussed elsewhere in this disclosure or in priority-claimed disclosures can also be added to the example shown in this figure.

FIG. 69 shows an electrode with a base and electroconductive protrusions, wherein protrusions closer to the center are less elastic or flexible. More specifically, FIG. 69 shows an example of a side view of a dry electrode for use on a hair-covered area comprising: a base 6901; and electroconductive protrusions which extend out from the base; wherein protrusions, including 6902, which are closer to the center of the base and/or a midline axis of the base have a first average elasticity and/or flexibility level; wherein protrusions, including 6903, which are farther from the center of the base and/or a midline axis of the base have a second average elasticity and/or flexibility level, and wherein the second average elasticity and/or flexibility level is greater than the first average elasticity and/or flexibility level. Relevant variations and claim limiting specifications discussed elsewhere in this disclosure or in priority-claimed disclosures can also be added to the example shown in this figure.

FIG. 70 shows an electrode with a base and electroconductive protrusions, wherein protrusions closer to the center are more elastic or flexible. More specifically, FIG. 70 shows an example of a side view of a dry electrode for use on a hair-covered area comprising: a base 7001; and electroconductive protrusions which extend out from the base; wherein protrusions, including 7002, which are closer to the center of the base and/or a midline axis of the base have a first average elasticity and/or flexibility level; wherein protrusions, including 7003, which are farther from the center of the base and/or a midline axis of the base have a second average elasticity and/or flexibility level, and wherein the second average elasticity and/or flexibility level is less than the first average elasticity and/or flexibility level. Relevant variations and claim limiting specifications discussed elsewhere in this disclosure or in priority-claimed disclosures can also be added to the example shown in this figure.

FIG. 71 shows an electrode with a base and electroconductive protrusions, wherein protrusions are evenly-distributed. More specifically, FIG. 71 shows an example of a bottom-up (e.g. distal-to-proximal) view of a dry electrode for use on a hair-covered area comprising: a base 7101; and electroconductive protrusions, including 7102, which extend out from the base, wherein protrusions are evenly-distributed (e.g. evenly-spaced) across the base. Relevant variations and claim limiting specifications discussed elsewhere in this disclosure or in priority-claimed disclosures can also be added to the example shown in this figure.

FIG. 72 shows an electrode with a base and electroconductive protrusions, wherein protrusions are around rings. More specifically, FIG. 72 shows an example of a bottom-up (e.g. distal-to-proximal) view of a dry electrode for use on a hair-covered area comprising: a base 7201; and electroconductive protrusions, including 7202, which extend out from the base, wherein protrusions are distributed along nested (e.g. concentric) rings of protrusions on the base. Relevant variations and claim limiting specifications discussed elsewhere in this disclosure or in priority-claimed disclosures can also be added to the example shown in this figure.

FIG. 73 shows an electrode with a base and electroconductive protrusions, wherein protrusions are along radial spokes. More specifically, FIG. 73 shows an example of a bottom-up (e.g. distal-to-proximal) view of a dry electrode for use on a hair-covered area comprising: a base 7301; and electroconductive protrusions, including 7302, which extend out from the base, wherein protrusions are distributed along virtual radial spokes extending out from the center of the base. Relevant variations and claim limiting specifications discussed elsewhere in this disclosure or in priority-claimed disclosures can also be added to the example shown in this figure.

FIG. 74 shows an electrode with a base and electroconductive protrusions, wherein protrusions are in a hub-and-spoke array. More specifically, FIG. 74 shows an example of a bottom-up (e.g. distal-to-proximal) view of a dry electrode for use on a hair-covered area comprising: a base 7401; and electroconductive protrusions, including 7402, which extend out from the base, wherein protrusions are distributed in a hub-and-spoke array on the base. Relevant variations and claim limiting specifications discussed elsewhere in this disclosure or in priority-claimed disclosures can also be added to the example shown in this figure.

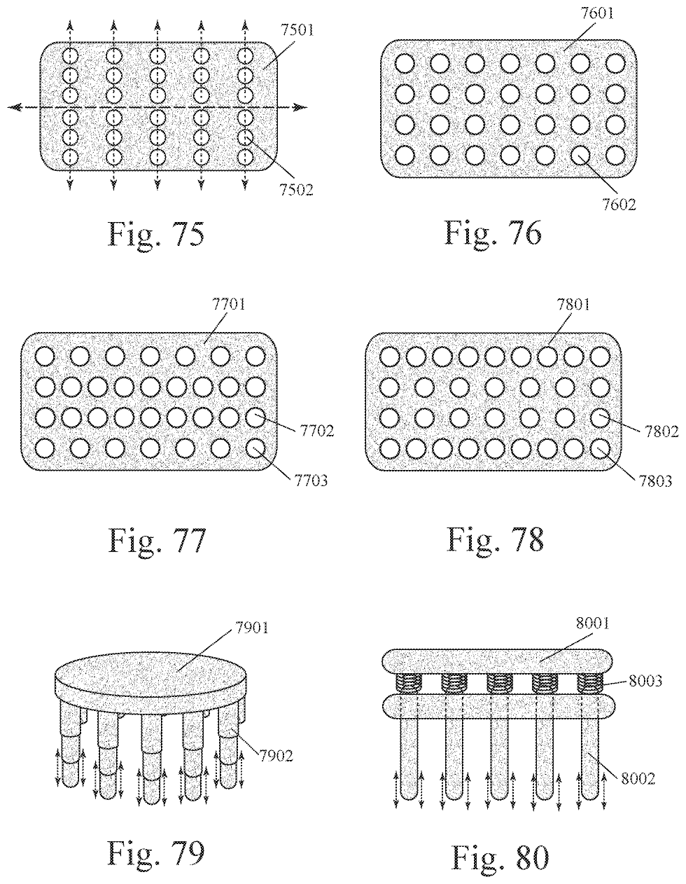

FIG. 75 shows an electrode with a base and electroconductive protrusions, wherein protrusions are along spokes extending out from a midline. More specifically, FIG. 75 shows an example of a bottom-up (e.g. distal-to-proximal) view of a dry electrode for use on a hair-covered area comprising: a base 7501; and electroconductive protrusions, including 7502, which extend out from the base, wherein protrusions are distributed along virtual spokes extending out from a midline of the base. Relevant variations and claim limiting specifications discussed elsewhere in this disclosure or in priority-claimed disclosures can also be added to the example shown in this figure.

FIG. 76 shows an electrode with a base and electroconductive protrusions, wherein protrusions are in a row-and-column array. More specifically, FIG. 76 shows an example of a bottom-up (e.g. distal-to-proximal) view of a dry electrode for use on a hair-covered area comprising: a base 7601; and electroconductive protrusions, including 7602, which extend out from the base, wherein protrusions are distributed in a row-and-column array on the base. Relevant variations and claim limiting specifications discussed elsewhere in this disclosure or in priority-claimed disclosures can also be added to the example shown in this figure.

FIG. 77 shows an electrode with a base and electroconductive protrusions, wherein protrusions closer to the center are closer together. More specifically, FIG. 77 shows an example of a bottom-up (e.g. distal-to-proximal) view of a dry electrode for use on a hair-covered area comprising: a base 7701; and electroconductive protrusions which extend out from the base; wherein protrusions, including 7702, which are closer to the center of the base and/or a midline axis of the base are a first average distance from each other; wherein protrusions, including 7703, which are farther from the center of the base and/or a midline axis of the base are a second average distance from each other, and wherein the second average distance is greater than the first average distance. Relevant variations and claim limiting specifications discussed elsewhere in this disclosure or in priority-claimed disclosures can also be added to the example shown in this figure.

FIG. 78 shows an electrode with a base and electroconductive protrusions, wherein protrusions closer to the center are farther apart. More specifically, FIG. 78 shows an example of a bottom-up (e.g. distal-to-proximal) view of a dry electrode for use on a hair-covered area comprising: a base 7801; and electroconductive protrusions which extend out from the base; wherein protrusions, including 7802, which are closer to the center of the base and/or a midline axis of the base are a first average distance from each other; wherein protrusions, including 7803, which are farther from the center of the base and/or a midline axis of the base are a second average distance from each other, and wherein the second average distance is less than the first average distance. Relevant variations and claim limiting specifications discussed elsewhere in this disclosure or in priority-claimed disclosures can also be added to the example shown in this figure.

FIG. 79 shows an electrode with a base and telescoping electroconductive protrusions. More specifically, FIG. 79 shows an example of an oblique side view of a dry electrode for use on a hair-covered area comprising: a base 7901; and telescoping electroconductive protrusions, including 7902, which extend out from the base. Relevant variations and claim limiting specifications discussed elsewhere in this disclosure or in priority-claimed disclosures can also be added to the example shown in this figure.

FIG. 80 shows an electrode with a base and individually spring-loaded electroconductive protrusions. More specifically, FIG. 80 shows an example of a side view of a dry electrode for use on a hair-covered area comprising: a base 8001; and electroconductive protrusions, including 8002, which extend out from the base, wherein individual protrusions are individually spring loaded (e.g. pushed toward the surface of a person's head by a spring) by individual springs, including 8003. Relevant variations and claim limiting specifications discussed elsewhere in this disclosure or in priority-claimed disclosures can also be added to the example shown in this figure.

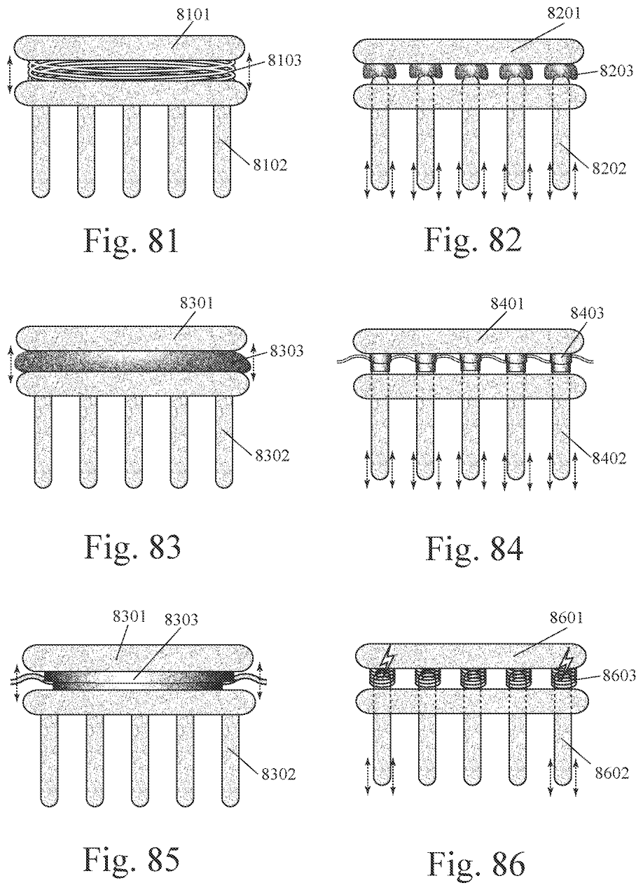

FIG. 81 shows an electrode with a base and collectively spring-loaded electroconductive protrusions. More specifically, FIG. 81 shows an example of a side view of a dry electrode for use on a hair-covered area comprising: a base 8101; and electroconductive protrusions, including 8102, which extend out from the base, wherein protrusions are collectively spring loaded (e.g. pushed toward the surface of a person's head by a spring) by a common spring 8103. Relevant variations and claim limiting specifications discussed elsewhere in this disclosure or in priority-claimed disclosures can also be added to the example shown in this figure.

FIG. 82 shows an electrode with a base and electroconductive protrusions, wherein protrusions are individually pushed toward the surface of a person's head by compressible members. More specifically, FIG. 82 shows an example of a side view of a dry electrode for use on a hair-covered area comprising: a base 8201; and electroconductive protrusions, including 8202, which extend out from the base, wherein individual protrusions are individually pushed toward the surface of a person's head by individual compressible members (e.g. foam pieces or inflatable chambers), including 8203. Relevant variations and claim limiting specifications discussed elsewhere in this disclosure or in priority-claimed disclosures can also be added to the example shown in this figure.

FIG. 83 shows an electrode with a base and electroconductive protrusions, wherein protrusions are collectively pushed toward the surface of a person's head by a compressible member. More specifically, FIG. 83 shows an example of a side view of a dry electrode for use on a hair-covered area comprising: a base 8301; and electroconductive protrusions, including 8302, which extend out from the base, wherein protrusions are collectively pushed toward the surface of a person's head by a common compressible member (e.g. foam piece or inflatable chamber) 8303. Relevant variations and claim limiting specifications discussed elsewhere in this disclosure or in priority-claimed disclosures can also be added to the example shown in this figure.

FIG. 84 shows an electrode with a base and electroconductive protrusions, wherein protrusions are individually pushed toward the surface of a person's head by pneumatic or hydraulic mechanisms More specifically, FIG. 84 shows an example of a side view of a dry electrode for use on a hair-covered area comprising: a base 8401; and electroconductive protrusions, including 8402, which extend out from the base, wherein individual protrusions are individually pushed toward the surface of a person's head by individual pneumatic or hydraulic mechanisms, including 8403. Relevant variations and claim limiting specifications discussed elsewhere in this disclosure or in priority-claimed disclosures can also be added to the example shown in this figure.

FIG. 85 shows an electrode with a base and electroconductive protrusions, wherein protrusions are collectively pushed toward the surface of a person's head by a pneumatic or hydraulic mechanism. More specifically, FIG. 85 shows an example of a side view of a dry electrode for use on a hair-covered area comprising: a base 8501; and electroconductive protrusions, including 8502, which extend out from the base, wherein protrusions are collectively pushed toward the surface of a person's head by a common pneumatic or hydraulic mechanism 8503. Relevant variations and claim limiting specifications discussed elsewhere in this disclosure or in priority-claimed disclosures can also be added to the example shown in this figure.

FIG. 86 shows an electrode with a base and electroconductive protrusions, wherein protrusions are individually pushed toward the surface of a person's head by electromagnetic actuators. More specifically, FIG. 86 shows an example of a side view of a dry electrode for use on a hair-covered area comprising: a base 8601; and electroconductive protrusions, including 8602, which extend out from the base, wherein individual protrusions are individually pushed toward the surface of a person's head by individual electromagnetic actuators (e.g. solenoids), including 8603. Relevant variations and claim limiting specifications discussed elsewhere in this disclosure or in priority-claimed disclosures can also be added to the example shown in this figure.

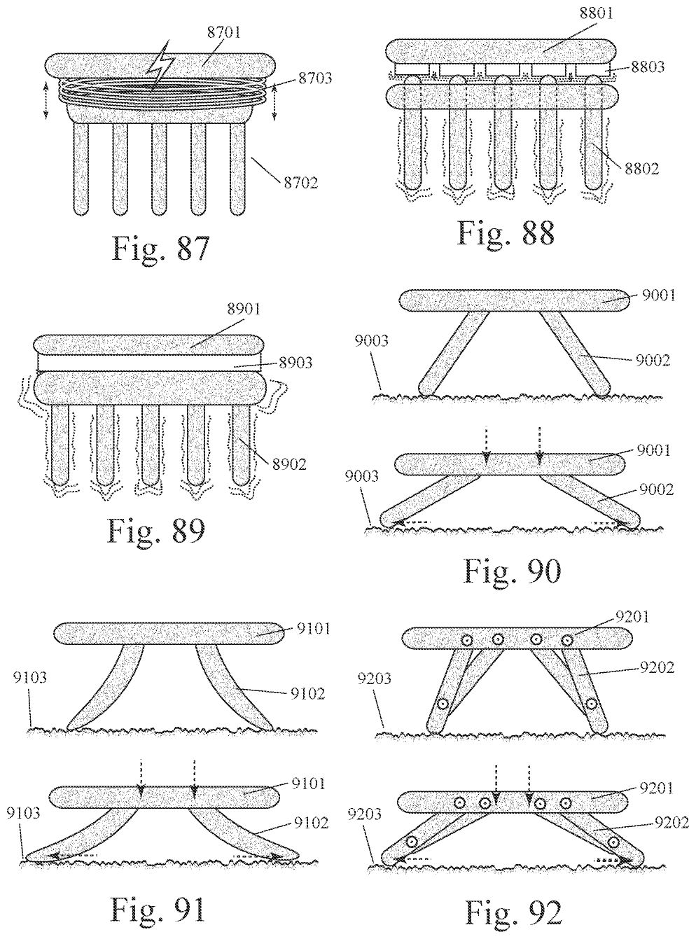

FIG. 87 shows an electrode with a base and electroconductive protrusions, wherein protrusions are collectively pushed toward the surface of a person's head by an electromagnetic actuator. More specifically, FIG. 87 shows an example of a side view of a dry electrode for use on a hair-covered area comprising: a base 8701; and electroconductive protrusions, including 8702, which extend out from the base, wherein protrusions are collectively pushed toward the surface of a person's head by a common electromagnetic actuator (e.g. solenoid) 8703. Relevant variations and claim limiting specifications discussed elsewhere in this disclosure or in priority-claimed disclosures can also be added to the example shown in this figure.

FIG. 88 shows an electrode with a base and electroconductive protrusions, wherein individual protrusions are vibrated and/or oscillated by individual electromagnetic actuators. More specifically, FIG. 88 shows an example of a side view of a dry electrode for use on a hair-covered area comprising: a base 8801; and electroconductive protrusions, including 8802, which extend out from the base, wherein individual protrusions are individually vibrated and/or oscillated along their longitudinal axes by individual electromagnetic actuators (e.g. solenoids), including 8803. Relevant variations and claim limiting specifications discussed elsewhere in this disclosure or in priority-claimed disclosures can also be added to the example shown in this figure.

FIG. 89 shows an electrode with a base and electroconductive protrusions, wherein protrusions are collectively vibrated and/or oscillated by an electromagnetic actuator. More specifically, FIG. 89 shows an example of a side view of a dry electrode for use on a hair-covered area comprising: a base 8901; and electroconductive protrusions, including 8902, which extend out from the base, wherein protrusions are collectively vibrated and/or oscillated along their longitudinal axes by a common electromagnetic actuator (e.g. solenoid) 8903. Relevant variations and claim limiting specifications discussed elsewhere in this disclosure or in priority-claimed disclosures can also be added to the example shown in this figure.

FIG. 90 shows an electrode with a base and electroconductive protrusions, wherein pushing protrusions toward the surface of a person's head spreads their tips farther apart. More specifically, the upper and lower portions of FIG. 90 show an example of two sequential side views of a dry electrode for use on a hair-covered area comprising: a base 9001; and electroconductive protrusions, including 9002, which extend out from the base, wherein the protrusions are configured so that pushing them toward the surface of the person's head 9003 causes their distal tips to move farther apart from each other (in a direction parallel to the surface of the person's head). Relevant variations and claim limiting specifications discussed elsewhere in this disclosure or in priority-claimed disclosures can also be added to the example shown in this figure.

FIG. 91 shows an electrode with a base and electroconductive protrusions, wherein protrusions are shaped so that pushing them toward the surface of a person's head spreads their tips farther apart. More specifically, the upper and lower portions of FIG. 91 show an example of two sequential side views of a dry electrode for use on a hair-covered area comprising: a base 9101; and electroconductive protrusions, including 9102, which extend out from the base, wherein the protrusions are shaped so that pushing them toward the surface of the person's head 9103 causes their distal tips to move farther apart from each other (in a direction parallel to the surface of the person's head). Relevant variations and claim limiting specifications discussed elsewhere in this disclosure or in priority-claimed disclosures can also be added to the example shown in this figure.

FIG. 92 shows an electrode with a base and articulated electroconductive protrusions, wherein pushing protrusions toward the surface of a person's head spreads their tips farther apart. More specifically, the upper and lower portions of FIG. 92 show an example of two sequential side views of a dry electrode for use on a hair-covered area comprising: a base 9201; and electroconductive protrusions, including 9202, which extend out from the base, wherein the protrusions are articulated so that pushing them toward the surface of the person's head 9203 causes their distal tips to move farther apart from each other (in a direction parallel to the surface of the person's head). Relevant variations and claim limiting specifications discussed elsewhere in this disclosure or in priority-claimed disclosures can also be added to the example shown in this figure.

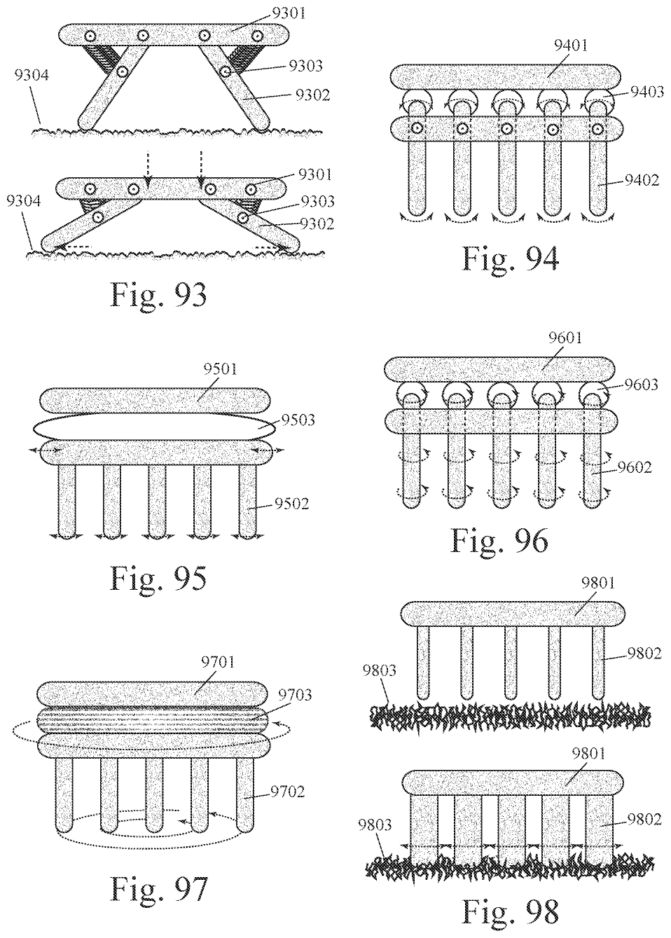

FIG. 93 shows an electrode with a base and electroconductive protrusions with hinges or joints, wherein pushing protrusions toward the surface of a person's head spreads their tips farther apart. More specifically, the upper and lower portions of FIG. 93 show an example of two sequential side views of a dry electrode for use on a hair-covered area comprising: a base 9301; and electroconductive protrusions, including 9302, which extend out from the base, wherein the protrusions have hinges or movable joints, including 9303, so that pushing them toward the surface of the person's head 9304, causes their distal tips to move farther apart from each other (in a direction parallel to the surface of the person's head). Relevant variations and claim limiting specifications discussed elsewhere in this disclosure or in priority-claimed disclosures can also be added to the example shown in this figure.

FIG. 94 shows an electrode with a base and electroconductive protrusions, wherein protrusions are individually vibrated and/or oscillated laterally by electromagnetic actuators. More specifically, FIG. 94 shows an example of a side view of a dry electrode for use on a hair-covered area comprising: a base 9401; and electroconductive protrusions, including 9402, which extend out from the base, wherein individual protrusions are individually vibrated and/or oscillated laterally (across the surface of a person's head) by individual electromagnetic actuators (e.g. solenoids), including 9403. Relevant variations and claim limiting specifications discussed elsewhere in this disclosure or in priority-claimed disclosures can also be added to the example shown in this figure.

FIG. 95 shows an electrode with a base and electroconductive protrusions, wherein protrusions are collectively vibrated and/or oscillated laterally by an electromagnetic actuator. More specifically, FIG. 95 shows an example of a side view of a dry electrode for use on a hair-covered area comprising: a base 9501; and electroconductive protrusions, including 9502, which extend out from the base, wherein protrusions are collectively vibrated and/or oscillated laterally (across the surface of a person's head) by a common electromagnetic actuator (e.g. solenoid) 9503. Relevant variations and claim limiting specifications discussed elsewhere in this disclosure or in priority-claimed disclosures can also be added to the example shown in this figure.

FIG. 96 shows an electrode with a base and electroconductive protrusions, wherein protrusions are individually rotated. More specifically, FIG. 96 shows an example of a side view of a dry electrode for use on a hair-covered area comprising: a base 9601; and electroconductive protrusions, including 9602, which extend out from the base, wherein individual protrusions are individually rotated (e.g. complete rotation or rotational oscillation) around their longitudinal axes by individual electromagnetic actuators, including 9603. Relevant variations and claim limiting specifications discussed elsewhere in this disclosure or in priority-claimed disclosures can also be added to the example shown in this figure.

FIG. 97 shows an electrode with a base and electroconductive protrusions, wherein protrusions are collectively rotated. More specifically, FIG. 97 shows an example of a side view of a dry electrode for use on a hair-covered area comprising: a base 9701; and electroconductive protrusions, including 9702, which extend out from the base, wherein protrusions are collectively rotated (e.g. complete rotation or rotational oscillation) around a central axis of the base by an electromagnetic actuator 9703. Relevant variations and claim limiting specifications discussed elsewhere in this disclosure or in priority-claimed disclosures can also be added to the example shown in this figure.

FIG. 98 shows an electrode with a base and electroconductive protrusions, wherein the cross-sectional size of protrusions is increased after their insertion between strands of hair. More specifically, the upper and lower portions of FIG. 98 show an example of two sequential side views of a dry electrode for use on a hair-covered area comprising: a base 9801; and electroconductive protrusions, including 9802, which extend out from the base, wherein protrusions have a first configuration with a first average cross-sectional size, wherein protrusions have a second configuration with a second cross-sectional size, wherein the second cross-sectional size is greater than the first cross-sectional size, and wherein the protrusions are changed from the first configuration to the second configuration after the protrusions have been inserted between strands of hair on the surface of a person's head 9803. Relevant variations and claim limiting specifications discussed elsewhere in this disclosure or in priority-claimed disclosures can also be added to the example shown in this figure.

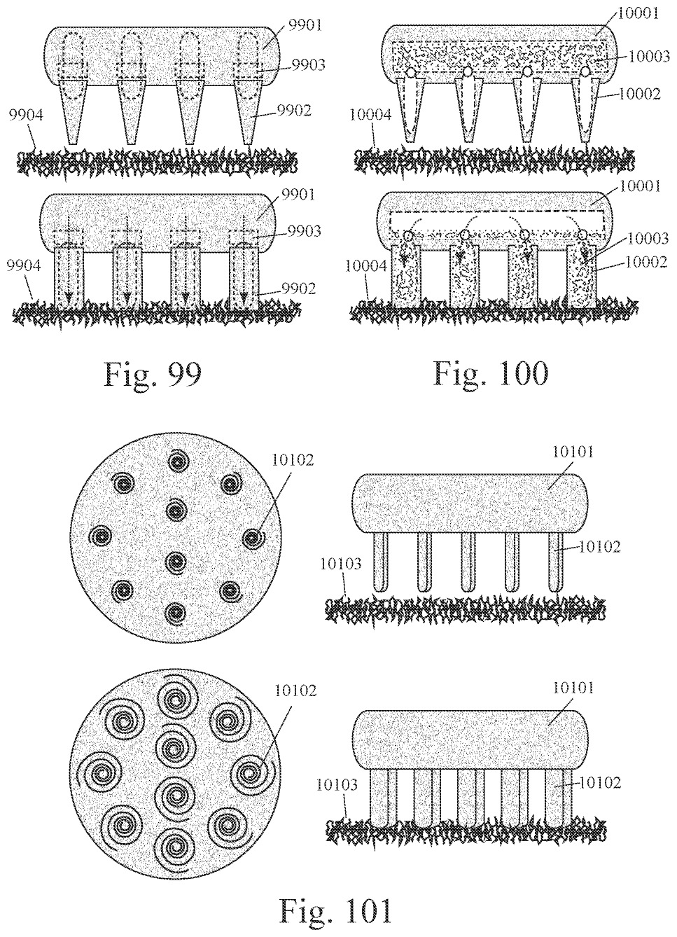

FIG. 99 shows an electrode with a base and electroconductive protrusions, wherein the cross-sectional size of protrusions is increased by electromagnetic actuators after their insertion between strands of hair. More specifically, the upper and lower portions of FIG. 99 show an example of two sequential side views of a dry electrode for use on a hair-covered area comprising: a base 9901; and electroconductive protrusions, including 9902, which extend out from the base, wherein protrusions have a first configuration with a first average cross-sectional size, wherein protrusions have a second configuration with a second cross-sectional size, wherein the second cross-sectional size is greater than the first cross-sectional size, and wherein the protrusions are changed from the first configuration to the second configuration after the protrusions have been inserted between strands of hair on the surface of a person's head 9904 by one or more electromagnetic actuators, including 9903. Relevant variations and claim limiting specifications discussed elsewhere in this disclosure or in priority-claimed disclosures can also be added to the example shown in this figure.

FIG. 100 shows an electrode with a base and electroconductive protrusions, wherein the cross-sectional size of protrusions is increased by inserting material into their interiors after insertion of the protrusions between strands of hair. More specifically, the upper and lower portions of FIG. 100 show an example of two sequential side views of a dry electrode for use on a hair-covered area comprising: a base 10001; and electroconductive protrusions, including 10002, which extend out from the base, wherein protrusions have a first configuration with a first average cross-sectional size, wherein protrusions have a second configuration with a second cross-sectional size, wherein the second cross-sectional size is greater than the first cross-sectional size, and wherein the protrusions are changed from the first configuration to the second configuration after the protrusions have been inserted between strands of hair on the surface of a person's head 10004 by insertion of material 10003 into the interiors of the protrusions. Relevant variations and claim limiting specifications discussed elsewhere in this disclosure or in priority-claimed disclosures can also be added to the example shown in this figure.

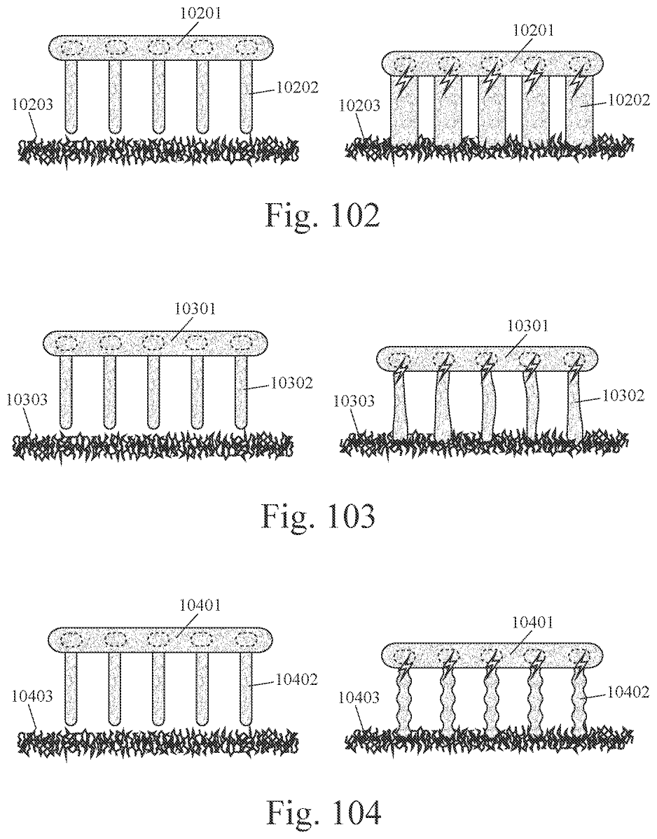

FIG. 101 shows an electrode with a base and electroconductive protrusions, wherein the cross-sectional size of protrusions is increased by uncoiling them after inserting them between strands of hair. More specifically, the upper and lower left-side portions of FIG. 101 show an example of two sequential bottom-up views and the upper and lower right-side portions of FIG. 101 show an example of two sequential side views of a dry electrode for use on a hair-covered area comprising: a base 10101; and electroconductive protrusions, including 10102, which extend out from the base, wherein protrusions have a first configuration with a first average cross-sectional size, wherein protrusions have a second configuration with a second cross-sectional size, wherein the second cross-sectional size is greater than the first cross-sectional size, and wherein the protrusions are changed from the first configuration to the second configuration after the protrusions have been inserted between strands of hair on the surface of a person's head 10103 by uncoiling the protrusions. Relevant variations and claim limiting specifications discussed elsewhere in this disclosure or in priority-claimed disclosures can also be added to the example shown in this figure.

FIG. 102 shows an electrode with a base and electroconductive protrusions, wherein the cross-sectional size of protrusions is increased by application of electrical energy after inserting the protrusions between strands of hair. More specifically, the left and right portions of FIG. 102 show an example of two sequential side views of a dry electrode for use on a hair-covered area comprising: a base 10201; and electroconductive protrusions, including 10202, which extend out from the base, wherein protrusions have a first configuration with a first average cross-sectional size, wherein protrusions have a second configuration with a second cross-sectional size, wherein the second cross-sectional size is greater than the first cross-sectional size, and wherein the protrusions are changed from the first configuration to the second configuration after the protrusions have been inserted between strands of hair on the surface of a person's head 10203 by the application of electrical energy. Relevant variations and claim limiting specifications discussed elsewhere in this disclosure or in priority-claimed disclosures can also be added to the example shown in this figure.

FIG. 103 shows an electrode with a base and electroconductive protrusions, wherein the durometer of protrusions is decreased after inserting them between strands of hair. More specifically, the left and right portions of FIG. 103 show an example of two sequential side views of a dry electrode for use on a hair-covered area comprising: a base 10301; and electroconductive protrusions, including 10302, which extend out from the base, wherein protrusions have a first configuration with a first durometer level, wherein protrusions have a second configuration with a second durometer level, wherein the second durometer level is less than the first durometer level, and wherein the protrusions are changed from the first configuration to the second configuration after the protrusions have been inserted between strands of hair on the surface of a person's head 10303 by the application of electrical energy. Relevant variations and claim limiting specifications discussed elsewhere in this disclosure or in priority-claimed disclosures can also be added to the example shown in this figure.

FIG. 104 shows an electrode with a base and electroconductive protrusions, wherein the flexibility or elasticity of protrusions is increased after inserting them between strands of hair. More specifically, the left and right portions of FIG. 104 show an example of two sequential side views of a dry electrode for use on a hair-covered area comprising: a base 10401; and electroconductive protrusions, including 10402, which extend out from the base, wherein protrusions have a first configuration with a first flexibility or elasticity level, wherein protrusions have a second configuration with a second flexibility or elasticity level, wherein the second flexibility or elasticity level is greater than the first flexibility or elasticity level, and wherein the protrusions are changed from the first configuration to the second configuration after the protrusions have been inserted between strands of hair on the surface of a person's head 10403 by the application of electrical energy. Relevant variations and claim limiting specifications discussed elsewhere in this disclosure or in priority-claimed disclosures can also be added to the example shown in this figure.

I claim:

1. An electrode configured for use on a hair-covered area of a person's head comprising:
a base, wherein the base has a first configuration with a first amount of curvature, wherein the base has a second configuration with a second amount of curvature, wherein the second amount is less than the first amount, and wherein the base is changed from the first configuration to the second configuration when the base is pressed onto a person's head; and
electroconductive protrusions which extend out from the base, wherein there is a first average distance between tips of the protrusions in the first configuration, wherein there is a second average distance between tips of the protrusions in the second configuration, and wherein the second average distance is greater than the first average distance.

2. The electrode in claim 1 wherein the base comprises a continuous flexible structure.

3. The electrode in claim 1 wherein the base comprises a plurality of connected moveable sections.

4. The electrode in claim 3 wherein the moveable sections are closer to being co-planar in the second configuration than in the first configuration.

5. An electrode configured for use on a hair-covered area of a person's head comprising:
a base; and
electroconductive protrusions which extend out from the base, wherein the protrusions are configured so that pushing the protrusions toward the surface of the person's head causes tips of the protrusions to move farther apart from each other.

6. The electrode in claim 5 wherein the protrusions are articulated so that pushing the protrusions toward the surface of the person's head causes tips of the protrusions to move farther apart from each other.

7. The electrode in claim 6 wherein the protrusions are articulated with hinges or movable joints so that pushing the protrusions toward the surface of the person's head causes tips of the protrusions to move farther apart from each other.

8. The electrode in claim 5 wherein a protrusion has a proximal section which is closer to the base and a distal section which is farther from the base, wherein the proximal section has a central longitudinal axis which intersects the plane of the base at a first outward-facing angle, and wherein the distal section has a central longitudinal axis whose virtual extension intersects the plane of the base at a second outward-facing angle.

9. The electrode in claim 8 wherein the second outward-facing angle is less than the first outward-facing angle.

10. The electrode in claim 8 wherein the second outward-facing angle is greater than the first outward-facing angle.

11. The electrode in claim 5 wherein protrusions which are closer to the center of the base connect to the base at a first average outward-facing angle, wherein protrusions which are farther from the center of the base connect to the base at a second average outward-facing angle, and wherein the second average outward-facing angle is less than the first average outward-facing angle.

12. The electrode in claim 5 wherein a protrusion is concave relative to a central longitudinal axis of the protrusion.

13. The electrode in claim 5 wherein a protrusion is convex relative to a central longitudinal axis of the protrusion.

14. The electrode in claim 5 wherein a protrusion has a proximal section which is closer to the base and a distal section which is farther from the base, wherein the proximal section is convex relative to a central longitudinal axis of the protrusion, and wherein the distal section is concave relative to a central longitudinal axis of the protrusion.

15. The electrode in claim 5 wherein a protrusion has a proximal section which is closer to the base and a distal section which is farther from the base, wherein the proximal section is concave relative to a central longitudinal axis of the protrusion, and wherein the distal section is convex relative to a central longitudinal axis of the protrusion.

16. The electrode in claim 5 wherein a protrusion has a proximal section which is closer to the base and a distal section which is farther from the base, and wherein the proximal section is bifurcated.

17. The electrode in claim 5 wherein a protrusion has a proximal section which is closer to the base and a distal section which is farther from the base, and wherein the distal section is bifurcated.

18. The electrode in claim 5 wherein a protrusion has a proximal section which is closer to the base, a distal section which is farther from the base, and a middle section between the proximal section and the distal section; and wherein the middle section is bifurcated.

19. An electrode configured for use on a hair-covered area of a person's head comprising:
a base; and
electroconductive protrusions which extend out from the base, wherein protrusions have a first configuration with a first average cross-sectional size, wherein protrusions have a second configuration with a second cross-sectional size, wherein the second cross-sectional size is greater than the first cross-sectional size, and wherein the protrusions are changed from the first configuration to the second configuration after the protrusions have been inserted between strands of hair on the surface of a person's head.

20. The electrode in claim 19 wherein protrusions are changed from the first configuration to the second configuration by a mechanism selected from the group consisting of: application of electrical energy to the protrusions; inserting solid matter into the interior of the protrusions; pumping a liquid or gas into the interior of the protrusions; and uncoiling the protrusions.
